(19) United States
(12) Patent Application Publication
(10) Pub. No.: US 2025/0372198 A1
(43) Pub. Date: Dec. 4, 2025

(54) SYSTEM AND METHOD FOR HIERARCHICAL TUMOR IMMUNE MICROENVIRONMENT EPIGENETIC DECONVOLUTION

(71) Applicant: The Trustees of Dartmouth College, Hanover, NH (US)

(72) Inventors: Brock C. Christensen, Lebanon, NH (US); Lucas A. Salas, Lebanon, NH (US); Ze Zhang, Hanover, NH (US); Karl T. Kelsey, Brookline, MA (US); John K. Wiencke, San Francisco, CA (US); Devin C. Koestler, Shawnee, KS (US)

(21) Appl. No.: 18/854,472

(22) PCT Filed: Feb. 6, 2023
(86) PCT No.: PCT/US2023/012438

§ 371 Date: Oct. 4, 2024

Related U.S. Application Data
(60) Provisional application No. 63/327,985, filed on Apr. 6, 2022.

(51) Int. Cl. G16B 20/00; G16B 40/20; G16H 50/20

(52) U.S. Cl. CPC G16B 20/00; G16B 40/20; G16H 50/20

(57) ABSTRACT

A system and method for determining a cancerous condition based upon at least one DNA sample is provided. An interface provides data related to DNA methylation for the sample, the data including related information about the sample. A processor, responsive to the interface, identifies the data related to the DNA methylation of the sample and accesses a data store containing a library of DNA methylation information related to each of tumor, immune, and angiogenic microenvironment components. A deconvolution process, relative to the DNA sample and the DNA methylation information, then determines association with one or more components from the sample. Illustratively, the library can define a plurality of layers of information associated with aspects of the cancerous condition relative to microenvironment components thereof. One or more components can define a tumor-type-specific hierarchical model related to a plurality of immune cell types that are subject to the deconvolution process.

### STATEMENT REGARDING FEDERALLY SPONSORED RESEARCH OR DEVELOPMENT

This invention was made with U.S. government support under Grant Numbers W81XWH-20-1-0778 awarded by the U.S. Congressionally Directed Medical Research Programs (CDMRP)/Department of Defense (DOD), P20GM104416/8299 awarded by the U.S. National Institute of General Medical Sciences (NIGMS) and R01 CA216265 awarded by the National Institutes of Health (NIH)/National Cancer Institute (NCI). The government has certain rights in this invention.

### FIELD OF THE INVENTION

This invention relates to systems and methods for diagnosis of cancerous conditions from cellular samples based upon deconvolution of DNA methylation data.

### BACKGROUND OF THE INVENTION

Beyond clonally-derived tumor cells, abundant and heterogenous cells that harbor these tumor cells constitute the tumor microenvironment (TME). As known in the literature, the TME plays an essential role in tumor differentiation, growth, and invasion. As also known in the literature, the TME comprises a spectrum of cell types responsible for immune and angiogenic responses. When antitumor immune responses are triggered, inflammatory cells populate the TME, including natural killer (NK) cells, active cytotoxic CD8 T cells, memory CD4 T cells, pro-inflammatory macrophages, and dendritic cells (DC). In contrast, a TME that contributes to functional evasion of tumor immune response includes Foxp3+ regulatory T cells (Tregs), exhausted CD8 T cells, inactive macrophages, and myeloid-derived suppressor cells (MDSCs). Non-tumor stromal cells and endothelial cells remodel the angiogenic microenvironment to support tumor growth and invasion. Also, the plasticity of epithelial cells plays a critical role in tumor progression. The dynamic interactions between tumor cells and other cells in their microenvironment can pro-mote tumor progression.

Tumor immune subtypes can be identified based on immunological gene expression profiling (See Wang H, Li S, Wang Q, Jin Z, Shao W, Gao Y, et al. Tumor immunological phenotype signature-based high-throughput screening for the discovery of combination immunotherapy compounds. Sci Adv. 2021.). Available on the WorldWideWeb at URL address, https://doi.org/10.1126/sciadv.abd7851. Tumors that are highly characterized by pro-inflammatory cytokines and T cell infiltration, i.e., immunologically hot tumors, have a better response rate to immune checkpoint inhibitors compared to immunologically cold tumors, which have a relatively low level of immune cell infiltration. However, the binary classification of hot and cold tumors oversimplifies the broader underlying immune landscape in TME. In the angiogenic microenvironment, tumors that are inclined to promote endothelial cell proliferation by producing vascular endothelial growth factor (VEGF) to develop new blood vessels can be targeted by angiogenesis inhibitors (See Sewduth R, Santoro M M. “Decoding” angiogenesis: new facets controlling endothelial cell behavior. Front Physiol. 2016; 7:306.), e.g., cancers of the lung, kidney, breast, colon, and rectum. Thus, understanding the heterogeneity of TME can guide therapy response and prognosis. See Labani-Motlagh A, Ashja-Mahdavi M, Loskog A. The tumor microenvironment: a milieu hindering and obstructing antitumor immune responses. Front Immunol. 2020; 11:940.

Gene expression and DNA methylation have been used to estimate cell composition in complex mixtures and include both reference-based and reference-free methods. CIBERSORT is a known and prominent reference-based method developed for deconvolving immune cell types using mRNA expression data. See Newman A M, Liu C L, Green M R, Gentles A J, Feng W, Xu Y, et al. Robust enumeration of cell subsets from tissue expression profiles. Nat Methods. 2015; 12(5):453-7. The accuracy of cell composition estimates using gene expression approaches is limited by variability in cell-specific gene expression across cells and the feature-space of gene expression data. DNA methylation is an epigenetic modification associated with gene regulation and is essential to lineage specification in development to establish and preserve cellular identity. See Bogdanovic O, Lister R. DNA methylation and the preservation of cell identity. Curr Opin Genet Dev. 2017; 46:9-14. There are three notable advantages to reference-based DNA methylation methods compared with RNA-based approaches in estimating cell composition. First, DNA is more stable than RNA. Second, the covalent addition of a methyl group to a cytosine is binary, tracking with cell count. Third, as recognized in the literature, using standard measurement approaches, the feature space to define reference profiles of cell-specific DNA methylation is at least 40-fold that of the typical gene expression feature space and can be up to 2000-fold higher. Extended libraries for reference-based DNA methylation deconvolution have been created, which result in improved accuracy and performance for peripheral blood immune cell deconvolution. See Salas L A, Zhang Z, Koestler D C, Butler R A, Hansen H M, Molinaro A M, et al. Enhanced cell deconvolution of peripheral blood using DNA methylation for high-resolution immune profiling. Nat Commun. 2022; 13(1):761; and Salas L A, Koestler D C, Butler R A, Hansen H M, Wiencke J K, Kelsey K T, et al. An optimized library for reference-based deconvolution of whole-blood biospecimens as saved using the Illumina HumanMethylationEPIC beadarray. Genome Biol. 2018; 19(1):64. By way of useful background information, see also commonly assigned U.S. patent application Ser. No. 17/670,346, entitled ENHANCED DNA METHYLATION LIBRARY FOR DECONVOLUTING PERIPHERAL BLOOD, filed Feb. 11, 2022, the teaching of which are incorporated by reference. Tissue-specific reference-based libraries have also been developed to infer cell-type composition in the brain, breast, and skin Salas L A, Lundgren S N, Browne E P, Punska E C, Anderton D L, Karagas M R, et al. Prediagnostic breast milk DNA methylation alterations in women who develop breast cancer. Hum Mol Genet. 2020; 29(4):662-73; and Muse M E, Bergman D T, Salas L A, Tom L N, Tan J M, Laino A, et al. Genomescale DNA methylation analysis identifies repeat element alterations that modulate the genomic stability of melanocytic nevi. J Invest Dermatol. 2021. WorldWideWeb URL address, https://doi.org/10.1016/j.jid.2021.11.025.

Initial approaches to deconvolve the TME using DNA methylation have been described. MethylCIBERSORT and MethylResolver have succeeded in resolving 10 and 12 cell types, respectively. However, due to the complexity and heterogeneity of the cell types in the TME, existing methods lack accuracy, specificity, and detailed cell types. Both the MethylCIBERSORT and MethylResolver methods used data from cancer cell lines rather than data from primary cancer cells. This is potentially problematic for deconvolution as cancer cell lines harbor additional epigenetic alterations as compared to primary tumors. Also, instead of using organ-specific epithelial cell type DNA methylation signatures, MethylResolver used a universal standard reference for tumor purity estimation in all tumor types.

### SUMMARY OF THE INVENTION

This invention overcomes disadvantages of the prior art by providing a system and method that enhances the accuracy and utility of TME deconvolution based upon the use of a novel DNA methylation-based process/algorithm that employs a tumor-type-specific hierarchical model and broadens the number of immune cell types that are deconvolved. The system and method, termed herein, Hierarchical Tumor Immune Microenvironment Deconvolution (HiTIMED), uses deconvolution libraries specific to tumor type, identifying the most cell-discriminatory CpG sites for each cell type in each tumor type context, resulting in (e.g.) 12 libraries per tumor type. The system and method also organizes deconvolution into the three major tumor microenvironment components (tumor, angiogenic, immune), resulting in the ability to resolve a total of (e.g.) 17 cell types in the TME: tumor, epithelial, endothelial, stromal, basophil, eosinophil, neutrophil, monocyte, dendritic cell (DC), B naïve (Bnv), B memory (Bmem), CD4T naïve (CD4nv), CD4T memory (CD4mem), CD8T naïve (CD8nv), CD8T memory (CD8mem), T regulatory (Treg), and natural killer (NK) cells, in (e.g.) 20 carcinoma types. The ability of the illustrative HiTIMED to resolve tumor cellular composition with high resolution provides a better understanding of cell heterogeneity in the TME, and allows for the study of more complex relationships of the TME with etiologic exposures, patient outcomes, and response to treatment of patients.

Notably, cellular compositions of solid TME are heterogeneous, varying across patients and tumor types. High-resolution profiling of the TME cell composition is highly to understanding its biological and clinical implications. Prior TME gene expression and DNA methylation-based deconvolution approaches have been able to deconvolve major cell types. However, existing methods lack accuracy and specificity to tumor type and include limited cell types. The illustrative HiTIMED desirably provides a DNA methylation-based algorithm to estimate cell proportions in the TME with high resolution and accuracy. HiTIMED deconvolution is amenable to archival biospecimens providing high-resolution profiles enabling to study of clinical and biological implications of variation and composition of the TME.

In an illustrative embodiment, a system and method for determining a cancerous condition based upon at least one DNA sample of an individual is provided. An interface arrangement provides data related to DNA methylation for the sample, the data including related information about the sample. A processor, responsive to the interface, identifies the data related to the DNA methylation of the sample and accesses a data store containing a library of DNA methylation information related to each of tumor, immune, and angiogenic microenvironment components. A deconvolution process, relative to the DNA sample and the DNA methylation information, then determines association with one or more components from the sample. Illustratively, the library can define a plurality of layers of information associated with aspects of the cancerous condition relative to microenvironment components thereof. One or more components can define a tumor-type-specific hierarchical model related to a plurality of immune cell types that are subject to the deconvolution process. The deconvolution process can be arranged to resolve a plurality of cell types, in which the cell types can include at least one of tumor, epithelial, endothelial, stromal, basophil, eosinophil, neutrophil, monocyte, dendritic cell (DC), B naïve (Bnv), B memory (Bmem), CD4T naïve (CD4nv), CD4T memory (CD4mem), CD8T naïve (CD8nv), CD8T memory (CD8mem), T regulatory (Treg), and natural killer (NK) cells. Illustratively, the library is provided in a data store accessed over a network arrangement by the processor. The deconvolution process can be performed by a trained artificial intelligence (AI) process. The system and method can be used particularly, for diagnosing and guiding the treatment of cancerous medical conditions employing results generated thereby. The systems and method can, thus, be used to treat the medical cancerous conditions based on clinical judgment of a practitioner and available therapies targeting specific cell components. The steps of the system and method can be performed by a non-transitory computer-readable medium of program instructions operating on the processor.

### BRIEF DESCRIPTION OF THE DRAWINGS

The invention description below refers to the accompanying drawings, of which:

FIGS. 1A and 1B is a block diagram showing of Hierarchical Tumor Immune Microenvironment Deconvolution (HiTIMED) tumor-type-specific hierarchical model, library development, and cell projection, including, for each carcinoma type, 12 libraries in 6 hierarchical layers (Library L1-Library L6B) that are optimized to estimate cell proportions, in which the first layer uses a tumor-type-specific reference library to deconvolve the tumor cell fraction from other cell types (Library L1), the second layer uses a library to separate tumor, angiogenic, and immune components (Library L2), and the third through sixth layers use libraries to deconvolve angiogenic and immune cell subtypes (Library L3A-L6B);

FIG. 2 is a series of graphs showing how cell composition differs substantially and capturing sample heterogeneity using the illustrative HiTIMED projected proportions;

FIGS. 3A-3F are graphs showing how tumor microenvironment (TME) heterogeneity measured by HiTIMED impacts 5-year survival in cancer patients, wherein Kaplan-Meier survival curves with statistically significant hazard ratios from Cox proportion hazard models with age, gender, tumor stage, tumor proportion, and other cell-type proportions adjusted by comparing survival in higher than median value (High) to lower than or equal to median group (Low) for B memory, CD8T memory, dendritic cell, Tregs, epithelial, endothelial, and stromal cells in pan-cancer survival analyses;

FIG. 4A-4D are graphs showing immune/angiogenic hot and cold tumors are distinguished using HiTIMED-based PAM clustering, with FIGS. 4A-4B showing immune hot and cold subtype proportions by TCGA tumor type and comparisons of major HiTIMED-projected cells between immune hot and cold tumors, and FIGS. 4C-4D showing angiogenic hot and cold subtype proportions by TCGA tumor type and comparisons of major HiTIMED-projected cells between angiogenic hot and cold tumors;

FIGS. 5A-5C are graphs showing angiogenic hot and cold tumors impact 5-year survival curves in head and neck squamous cell carcinoma, thyroid carcinoma, and stomach carcinoma, in which hazard ratios are from Cox models adjusting for age, gender, and tumor stage;

FIGS. 6A-6D are plots showing how, independently of the tumor type, TCGA samples can be classified by HiTIMED immune hot and cold subtypes, angiogenic hot and cold subtypes, and immune and angiogenic hot and cold subtypes, in which Uniform Manifold Approximation and Projection for Dimension Reduction (UMAP) clustering is used to classify the samples based on the HiTIMED TME cell composition, colored by tumor type and the angiogenic/immune classification;

FIG. 7A-7C are plots showing EWAS output comparisons across three models, including Model 1 adjusted for age and gender, Model 2 adjusted for age, gender, and HiTIMED-projected tumor purity, and Model 3 adjusted for age, gender, HiTIMED-projected tumor purity, DC, CD8mem, Bmem, Treg, epithelial, endothelial, and stromal cell proportions. Delta betas larger than 0.3 and FDR smaller than 0.01 are used as the cut-off for statistically significant DMC identification;

FIG. 7D is a table comparing Models, Model 3 and Model 3 based on hypermethelated and hypomethylated measurements in an exemplary tumor;

FIGS. 7E-7G each show heatmaps with Manhattan distance clustering and colon cancer CIMP subtypes shaded are generated for each Model 1-Model 3, respectively;

FIGS. 8A-8E are graphs showing correlation between HiTIMED tumor and InfiniumPurify tumor by tumor type, in which HiTIMED projected tumor proportions are highly significantly correlated with the InfiniumPurify predicted tumor purities across tumor types;

FIGS. 9A and 9B are comparative heatmaps showing Methylation state of CpGs in the HiTIMED tumor specific library (L1) and InfiniumPurify default library between tumor and normal samples across cholangiocarcinoma, kidney papillary cell carcinoma, pancreatic adenocarcinoma, and stomach adenocarcinoma;

FIGS. 10A and 10B are graphs showing HiTIMED tumor purity vs InfiniumPurify tumor purity in thyroid carcinoma, in which a cluster of HiTIMED-predicted tumor purity low but InfiniumPurify-predicted high tumor is identified and colored in heatmaps, and HiTIMED tumor proportion in thyroid carcinoma shaded by invasive and non-invasive tumor type;

FIGS. 11A-11D are graphs showing HiTIMED tumor proportion vs other method predicted tumor proportion;

FIGS. 12A-12F are graphs showing HiTIMED immune cell proportions vs true immune cell proportions in artificial mixtures;

FIG. 13 is a graph showing HiTIMED T cell proportion vs true T cell proportion in artificial mixtures, in which T cell proportions correspond to the sum of CD4T naive and memory, CD8 naive and memory and T regulatory cells;

FIGS. 14A and 14B are graphs showing HiTIMED cell composition in human normal intestinal epithelium and umbilical vein endothelial cells, respectively;

FIG. 15A is a Venn diagram showing HiTIMED, MethyICIBERSORT, and MethylResolver cell type applicability;

FIGS. 15B and 15C are graphs showing performance comparison across HiTIMED, MethylCIBERSORT, and MethylResolver using artificial mixtures;

FIGS. 16A-16E is a sequence of graphs showing the distribution of the HiTIMED cell composition in TCGA tumors;

FIGS. 17A-17D are graphs and tables showing how Cell composition differs substantially, and capturing sample heterogeneity using HiTIMED-projected proportions, in which (e.g.) seventeen (17) cell types are captured for each sample by tumor type;

FIG. 18 is a sensitive analysis comparing outputs from two Cox models with or without cell type proportions adjusted in kidney clear cell carcinoma;

FIGS. 19A-19T are graphs showing Kaplan-Meier survival curves for HiTIMED cells estimates in TCGA tumors, in which hazard ratios are calculated from the Cox proportional hazard models with age, gender, and tumor proportion adjusted, and gender is not adjusted for gender-specific tumors;

FIG. 20A is a graph showing HiTIMED cell comparison;

FIGS. 20B-20E are graphs showing Kaplan-Meier survival curves across immune/angiogenic hot and cold tumors, in which P-values are calculated from the log-rank tests;

FIGS. 21A and 21B are graphs showing HiTIMED immune and angiogenic proportions across C1-C6 subtyped TCGA tumor, respectively;

FIG. 22 is a graph of HiTIMED cell comparisons between drug-sensitive and resistant metastasized colorectal cancer;

FIGS. 23A and 23B are graphs showing HiTIMED cell comparisons in triple-negative breast cancer w/without chemotherapy;

FIGS. 24A-24E are graphs and a chart showing performance comparison across iterations on CpGs selected in HiTIMED for immune and angiogenic cell projection;

FIG. 25 is a block diagram showing a generalized computing environment for performing the processes and steps of the system and method herein; and

FIG. 26 is a flow diagram of a generalized procedure for performing the system of method within the computing environment of FIG. 25.

### DETAILED DESCRIPTION

### I. Abbreviations

To assist the reader, the following abbreviations are used in the Specification and Drawings herein relative to the terms listed as follows:
TME: Tumor microenvironment;
DC: Dendritic cell;
Mono: Monocyte;
Bas: Basophil;
Eos: Eosinophil;
Neu: Neutrophil;
NK: Natural killer cell;
Bnv: B naïve cell;
Bmem: B memory cell;
CD4nv: CD4 naïve cell;
CD4mem: CD4 memory cell;
Treg: T regulatory cell;
CD8nv: CD8 naïve cell;
CD8mem: CD8 memory cell;
BLCA: Bladder urothelial carcinoma;
BRCA: Breast invasive carcinoma;
CESC: Cervical squamous cell carcinoma and endocervical adenocarcinoma;
CHOL: Cholangiocarcinoma;
COAD: Colon adenocarcinoma;
ESCA: Esophageal carcinoma;
HNSC: Head and neck squamous cell carcinoma;
KIRC: Kidney clear cell renal cell carcinoma;
LIHC: Liver hepatocellular carcinoma;
LUAD: Lung adenocarcinoma;
LUSC: Lung squamous cell carcinoma;
PAAD: Pancreatic adenocarcinoma;
PRAD: Prostate adenocarcinoma;
READ: Rectum adenocarcinoma;
STAD: Stomach adenocarcinoma;
THCA: Thyroid carcinoma; UCEC;
Uterine corpus endometrial carcinoma;
TCGA: The Cancer Genome Atlas; and
TNBC: Triple-negative breast cancer.

### II. Library Development and Technical Considerations

A. HiTIMED Tumor-type-specific Hierarchical Model, Library Development, and Cell Projection

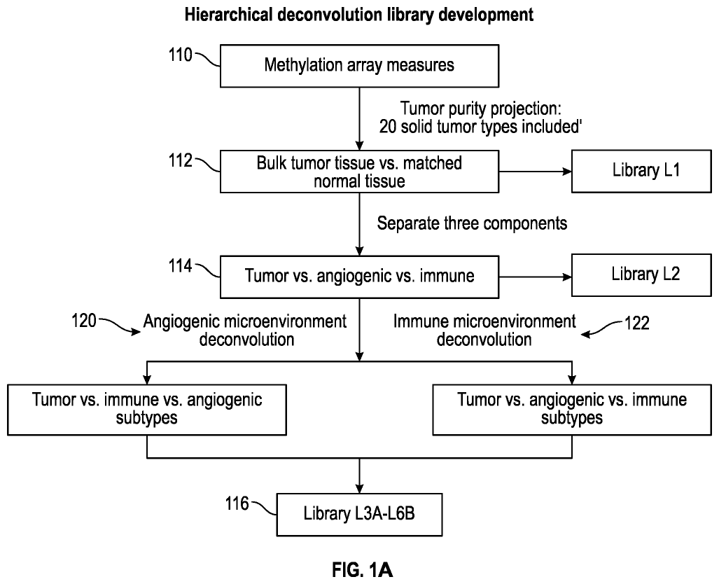

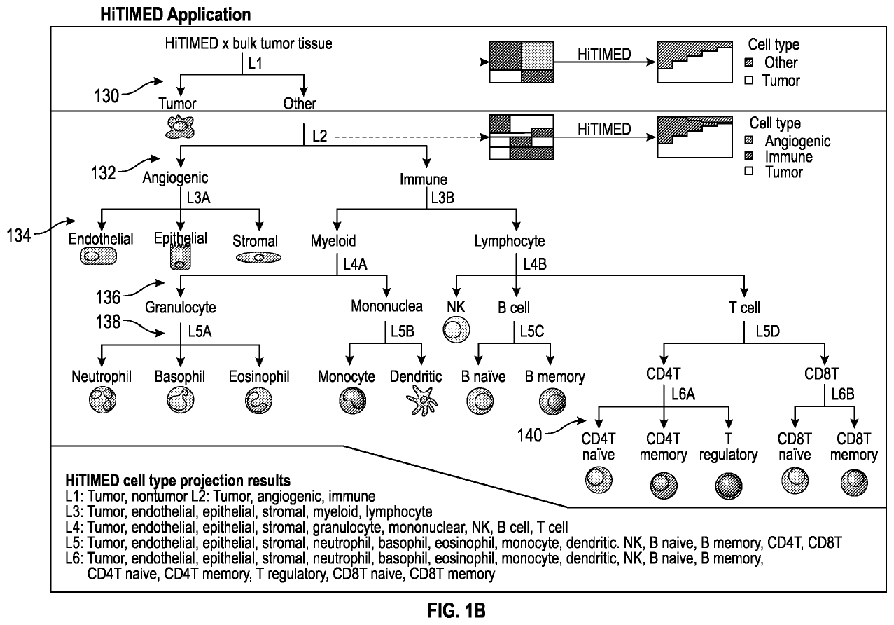

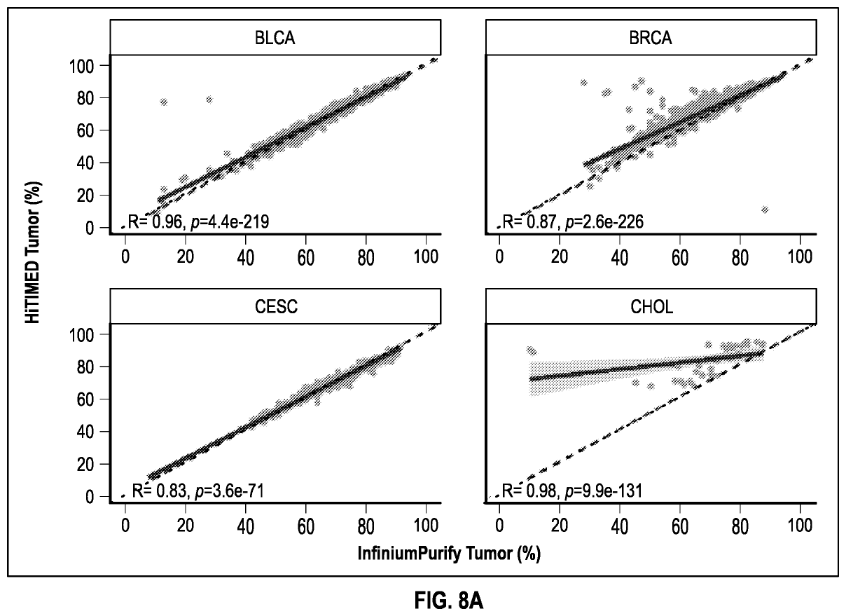

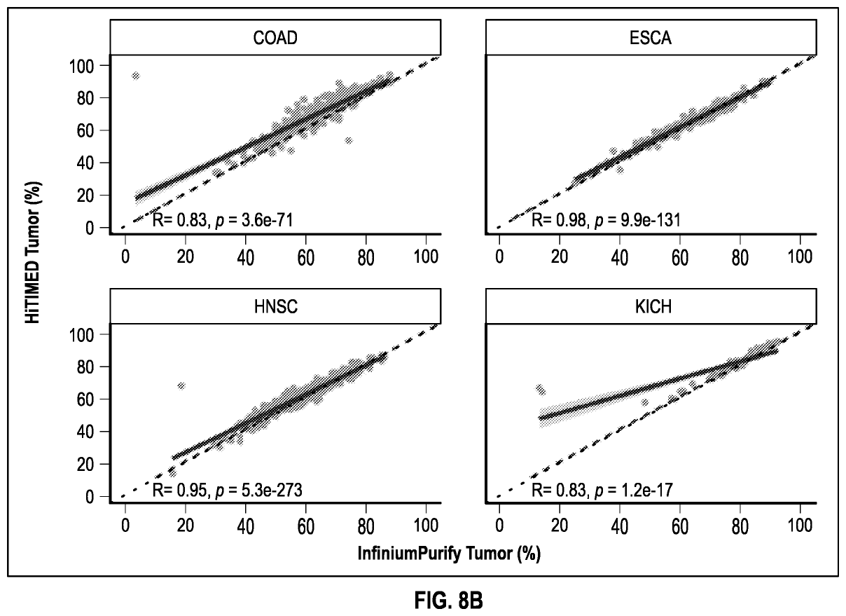

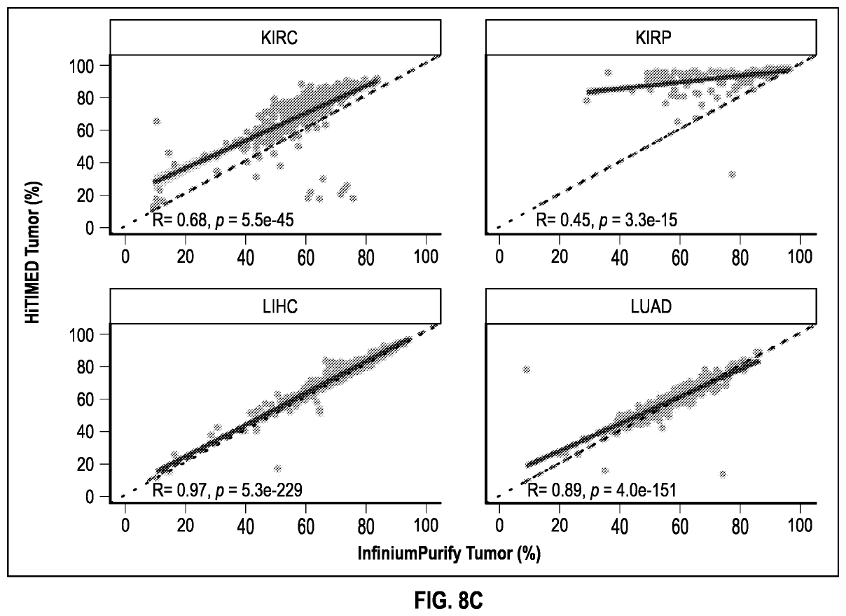

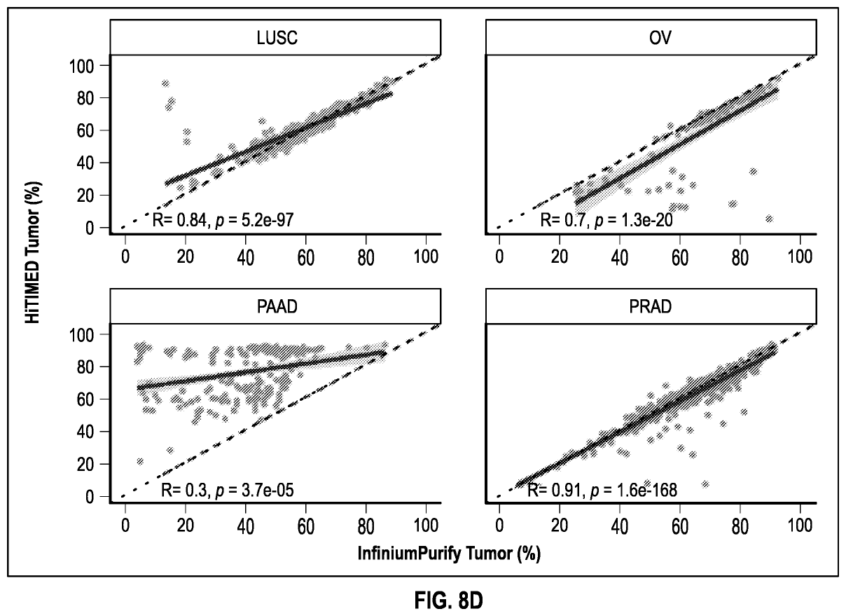

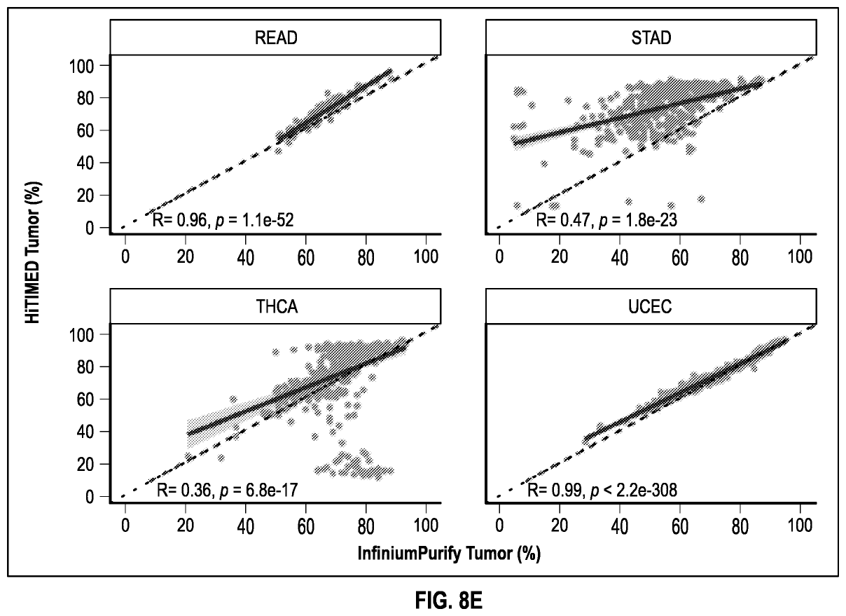

According to an exemplary embodiment of the system and method herein, HiTIMED employs a novel tumor-type-specific hierarchical model to deconvolve the TME. To develop HiTIMED, discovery data from (e.g.) 6726 samples is used, by way of non-limiting example, across 20 types of carcinomas and matched normal or normal-adjacent tissue. In addition, (e.g.) 26 samples for three angiogenic/non-immune cell types, and 61 samples for 13 immune cell types are included as shown generally in FIGS. 8A-8E. Twelve (12) libraries in (e.g.) six hierarchical layers are optimized for each carcinoma type to estimate cell proportions. The first layer (Library L1) 130 uses a tumor-type-specific reference library to deconvolve the tumor cell fraction from other cell types. Reference is, thus, made to FIGS. 1A and 1B, in which Library L1 is developed by identifying the top (e.g.) 1000 most informative differentially methylated CpG sites 110 from cancer-normal comparisons 112 using the InfiniumPurify pipeline. See also Zheng X, Zhang N, Wu H J, Wu H. Estimating and accounting for tumor purity in the analysis of DNA methylation data from cancer studies. Genome Biol. 2017; 18(1):17. To discern tumor, immune, and angiogenic cells 114, in the second layer 132, Library L2 and subsequent libraries 116 have been developed using the Meffil package (see Min J L, Hemani G, Davey Smith G, Relton C, Suderman M. Meffil: efficient normalization and analysis of very large DNA methylation datasets. Bioinformatics. 2018; 34(23):3983-9), which uses limma linear regression with empirical Bayes adjustment statistics to reduce methylation profiles to top (e.g.) 100 cell-type-specific hyper- and hypo-methylated CpGs. Then, two reference libraries in the third layer 134 of the hierarchical deconvolution are applied. Library L3A discerns the angiogenic microenvironment and deconvolves endothelial, epithelial, and stromal cell components. Library L3B separates lymphoid and myeloid cell fractions in the immune microenvironment 122. In the fourth layer 136, Library L4A distinguishes granulocytes and mononuclear cells under the myeloid lineage, and Library L4B separates NK, B, and T cells, in the lymphocyte lineage. In the fifth layer 138, Library L5A discerns neutrophils, basophils, and eosinophils, under the granulocyte lineage, and Library L5B discriminates monocyte and dendritic cells under the mononuclear cell lineage. Library L5C differentiates B naïve, and B memory cells under the B cell lineage, and Library L5D is developed to detect CD4T and CD8T cells under the T cell lineage. In the sixth layer 140, Library L6A recognizes CD4T naïve, CD4T memory, and T regulatory cells under the CD4T lineage, and Library L6B differentiates CD8T naïve and CD8T memory under the CD8T lineage.

Cell proportions in the tumor TME are projected hierarchically using the above-mentioned Libraries. In the first layer, tumor and nontumor proportions are predicted by the probability density of methylation levels of Library L1 CpGs using the InfiniumPurify pipeline. From the second layer to the sixth layer, Libraries L2 to L6B are used in conjunction with the constrained projection quadratic programming approach described by Houseman et al. (see Houseman E A, Accomando W P, Koestler D C, Christensen B C, Marsit C J, Nelson H H, et al. DNA methylation arrays as surrogate measures of cell mixture distribution. BMC Bioinformatics. 2012; 13:86) to project the proportions of angiogenic and immune cells in the nontumor component from the first layer hierarchically by weighting the lower layer cell projections to the higher layer cell projections. In this manner, (e.g.) twenty (20) sets of twelve (12) Libraries are identified—one for each type of carcinoma—to optimally deconvolve the TME. The HiTIMED deconvolution function in the HiTIMED package is thus created to deconvolve the TMEs with a user-specified tumor site and layer. The package is available on the WorldWideWeb at the URL address, https://github.com/SalasLab/HiTIMED.

### B. HiTIMED Validation

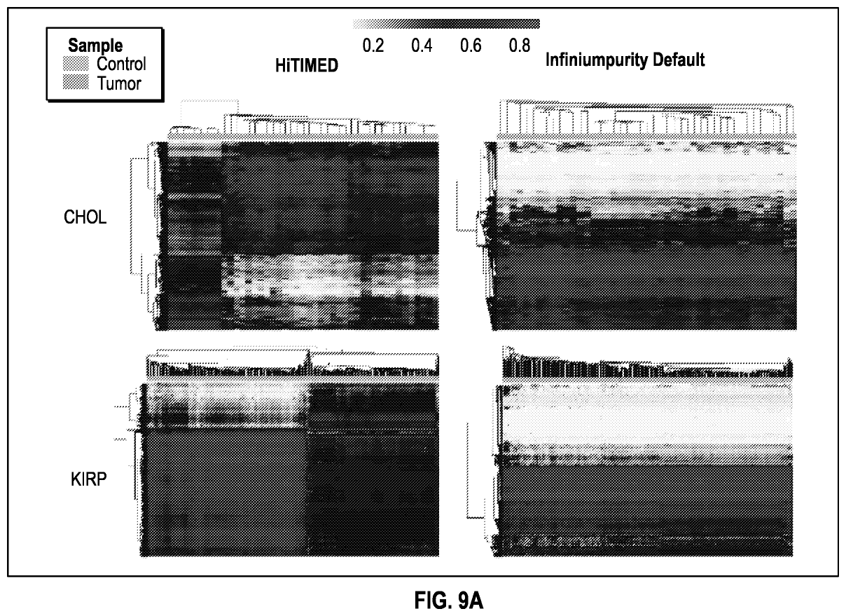

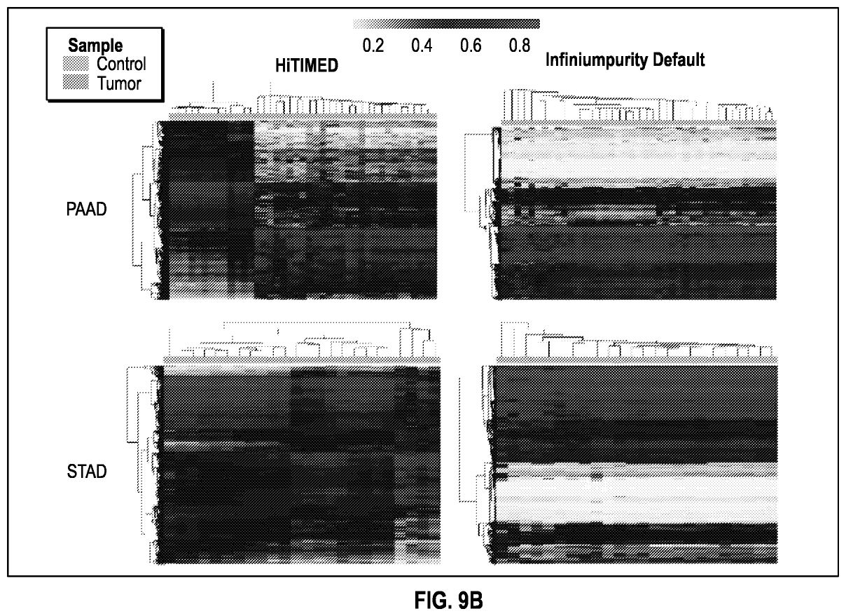

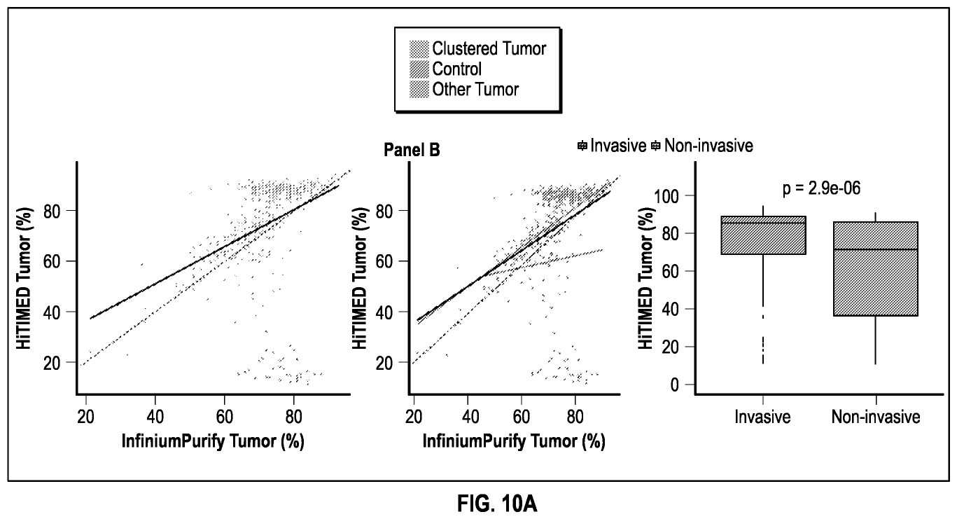

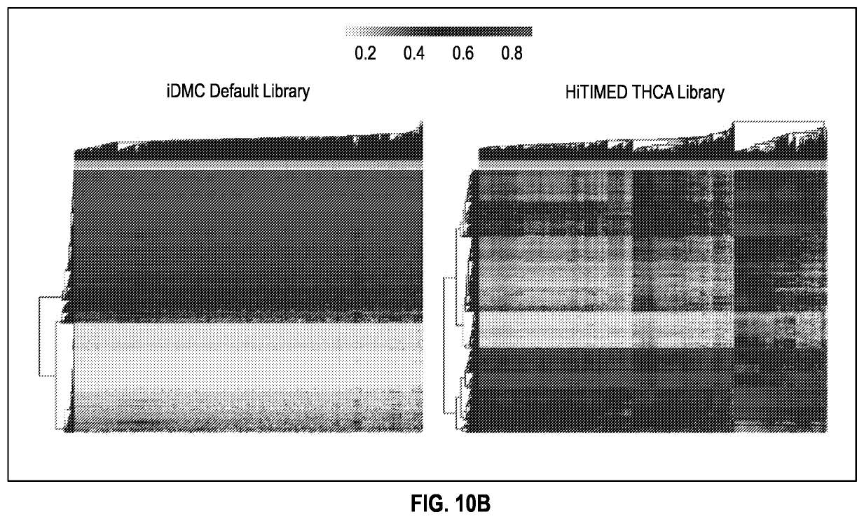

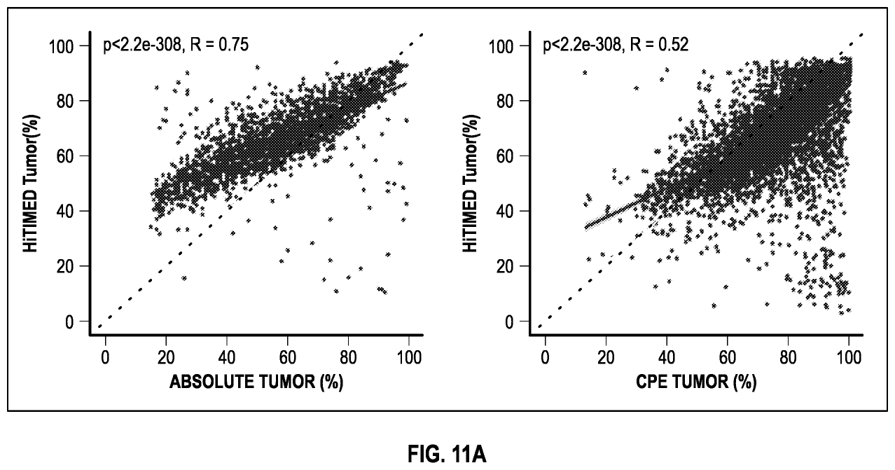

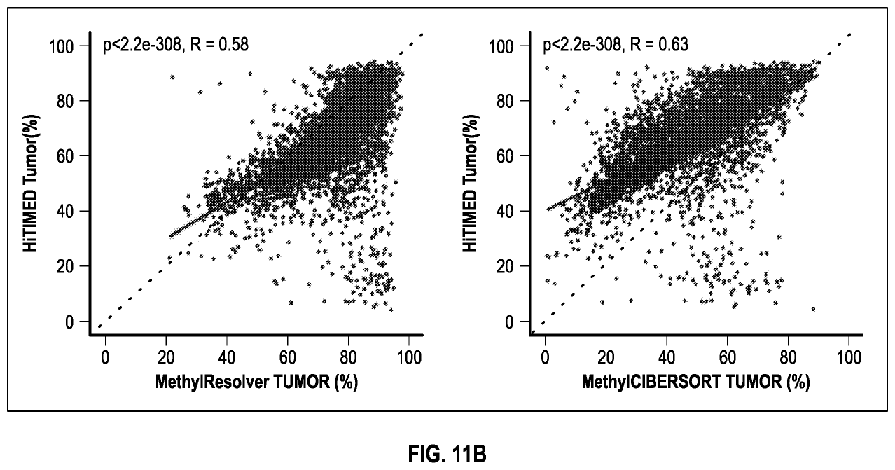

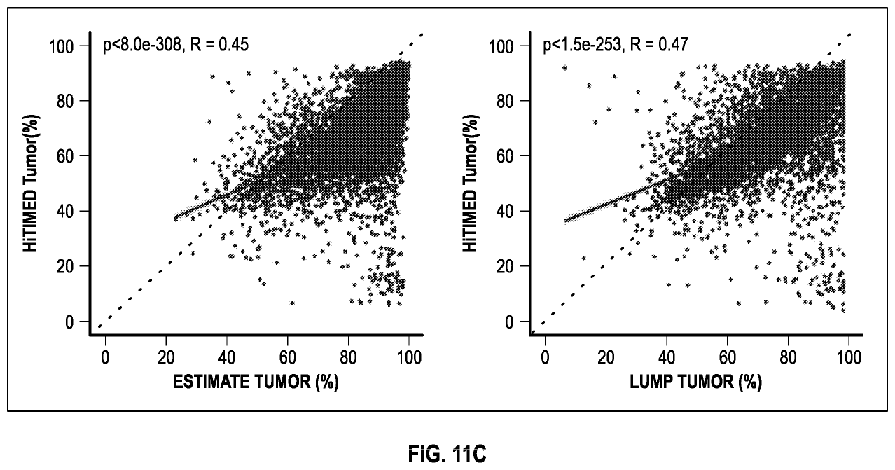

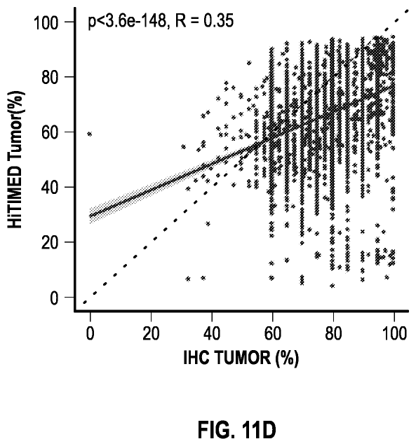

To validate tumor purity estimates from HiTIMED, the HiTIMED projected tumor cell proportion is compared with the existing tumor purity estimation methods on publicly available tumor data. InfiniumPurify is a methylation-based and validated method for tumor purity prediction. HiTIMED projected tumor proportions correlate significantly with the InfiniumPurify predicted tumor purities across tumor types (FIGS. 8A-8E). Although highly correlated for most tumor types, five tumor types demonstrate correlation coefficients less than 0.5 (i.e., cholangiocarcinoma, kidney papillary, pancreatic, stomach, and thyroid carcinoma). To further validate the system and method for those five tumor types, it has been shown that the HiTIMED tumor-specific library has a clearer methylation distinction between tumor and normal samples compared to the InfiniumPurify's default library for tumor purity estimation (See FIGS. 9A and 9B). Furthermore, among thyroid carcinomas, it has been observed that a cluster of tumors with lower tumor cell proportions from HiTIMED compared with InfiniumPurify. The depicted heatmaps demonstrate a more similar methylation state of the clustered tumors with controls compared to other tumors, which is not captured by InfiniumPurify (See FIG. 10A). Note that the cluster is predominantly composed of non-invasive follicular thyroid neoplasm with papillary-like nuclear features, and non-invasive follicular thyroid tumor purity is significantly lower than the invasive papillary thyroid carcinoma (See heatmaps in FIG. 10B). Several tumor purity estimation methods, including those that use data sources other than DNA methylation, have been compared to HiTIMED. These include several known techniques, including, methylation-based MethylCIBERSORT (See Chakravarthy A, Furness A, Joshi K, Ghorani E, Ford K, Ward M J, et al. Pancancer deconvolution of tumour composition using DNA methylation. Nat Commun. 2018; 9(1):3220.), MethylResolver (See Arneson D, Yang X, Wang K. MethylResolver—a method for deconvoluting bulk DNA methylation profiles into known and unknown cell contents. Commun Biol. 2020; 3(1):422.), LUMP (See Benelli M, Romagnoli D, Demichelis F. Tumor purity quantification by clonal DNA methylation signatures. Bioinformatics. 2018; 34(10):1642-9.), gene expression-based ESTIMATE (Yoshihara K, Shahmoradgoli M, Martinez E, Vegesna R, Kim H, Torres-Garcia W, et al. Inferring tumour purity and stromal and immune cell admixture from expression data. Nat Commun. 2013; 4:2612.), somatic copy-number-based ABSOLUTE (See Carter S L, Cibulskis K, Helman E, McKenna A, Shen H, Zack T, et al. Absolute quantification of somatic DNA alterations in human cancer. NatBiotechnol. 2012; 30(5):413-21.), image stain-based immunohistochemistry IHC, and consensus measurement of purity estimations (CPE) (See Aran D, Sirota M, Butte A J. Systematic pan-cancer analysis of tumour purity. Nat Commun. 2015; 6:8971.). The results have demonstrated significantly and highly correlated tumor cell projections with HiTIMED as compared to other established methods (See graphs of FIGS. 11A-11D). To validate the immune cell projections from HiTIMED, 12 immune cell artificial mixture samples are deconvolved, whose ground truth immune composition across 12 cell types is known

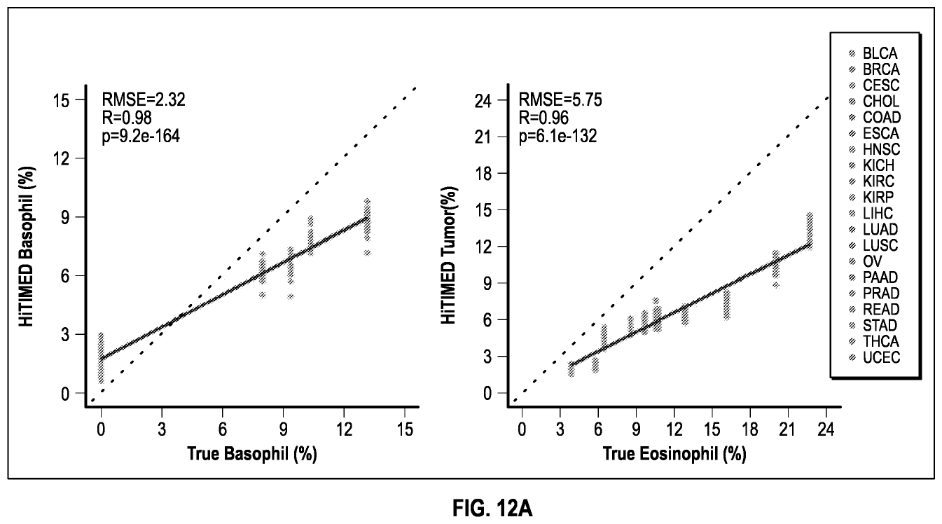

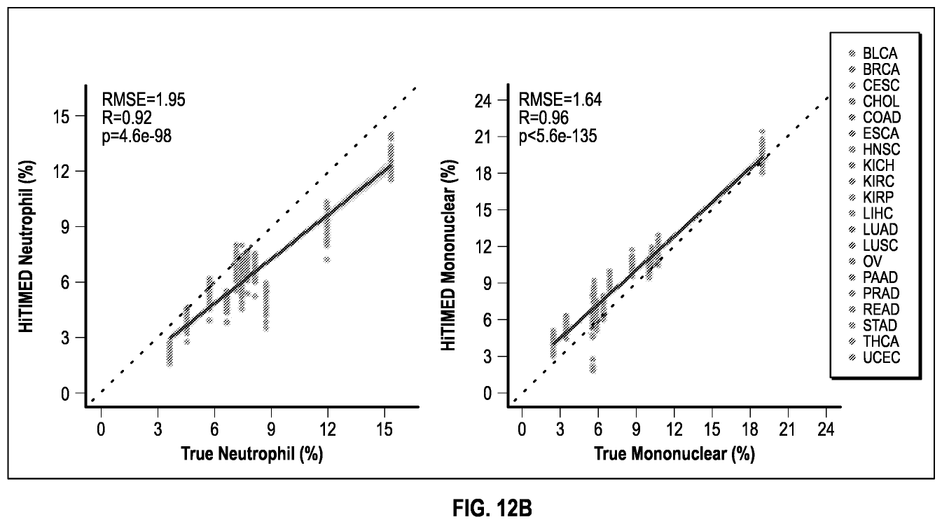

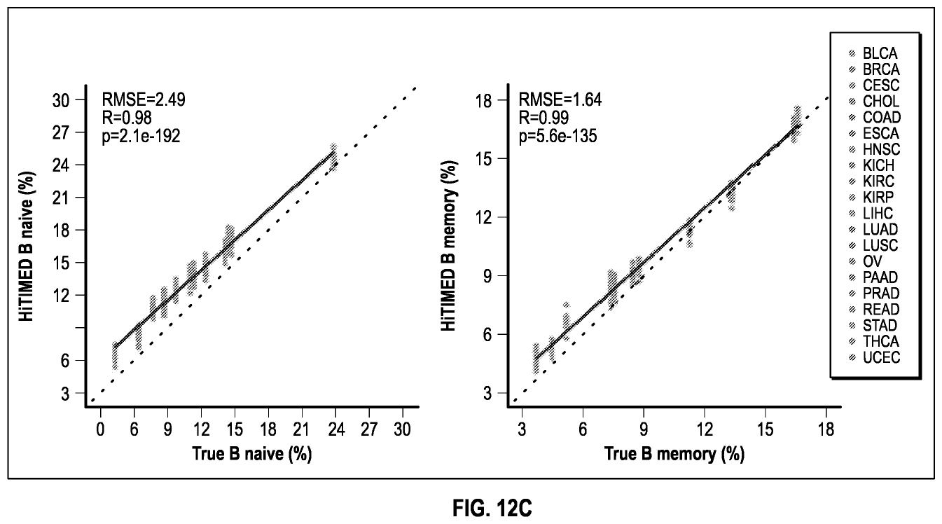

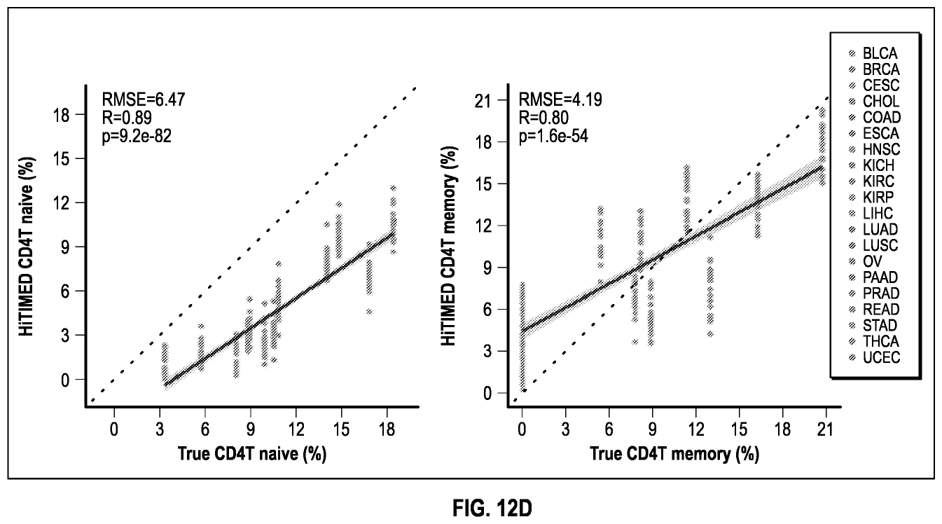

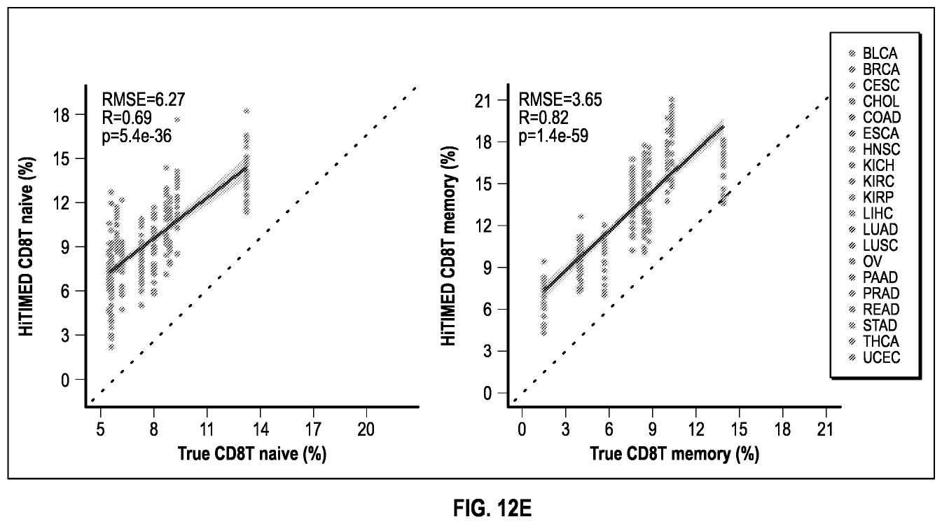

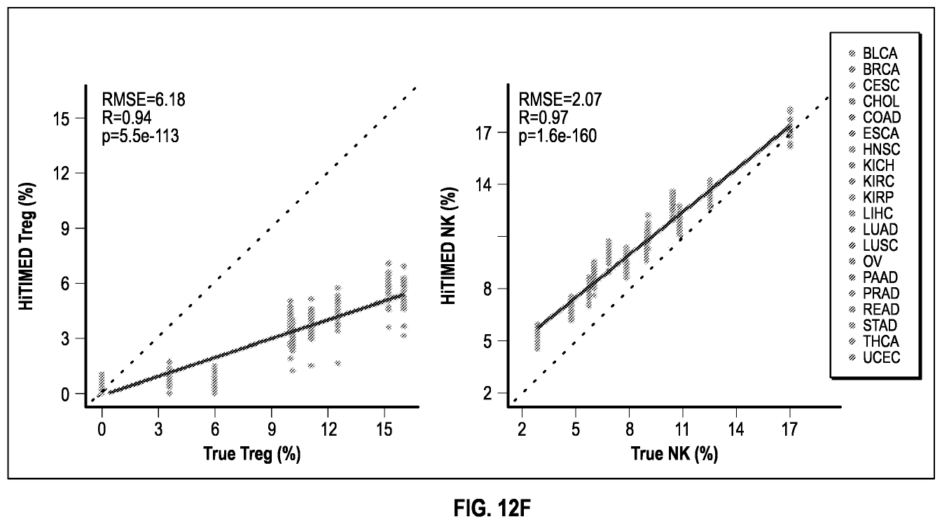

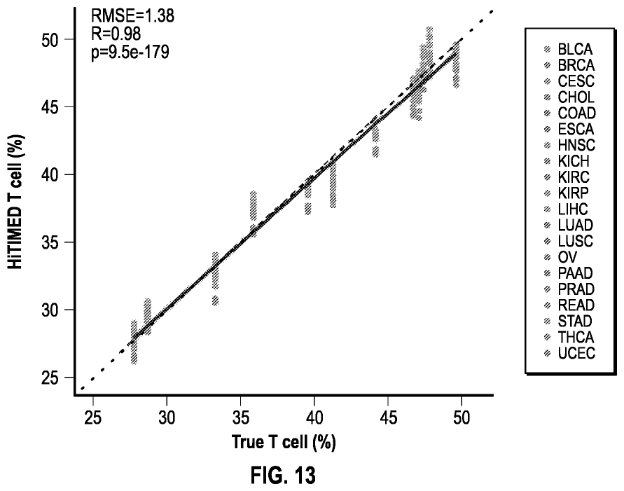

All 12 immune cells show a highly significant correlation between HiTIMED prediction and ground truth and low RMSE. 8 out of 12 cell types showed Pearson's correlation coefficients (R) over 0.90, and 11 out of 12 cell types showed R over 0.80 (See FIGS. 12A-12F). Although the depicted scatterplots demonstrated slight under-prediction for some CD4T cell subsets and slight over-prediction for some CD8T cell subsets, the HiTIMED prediction for total T cells is highly accurate (R=0.98, RMSE=1.38, FIG. 13)).

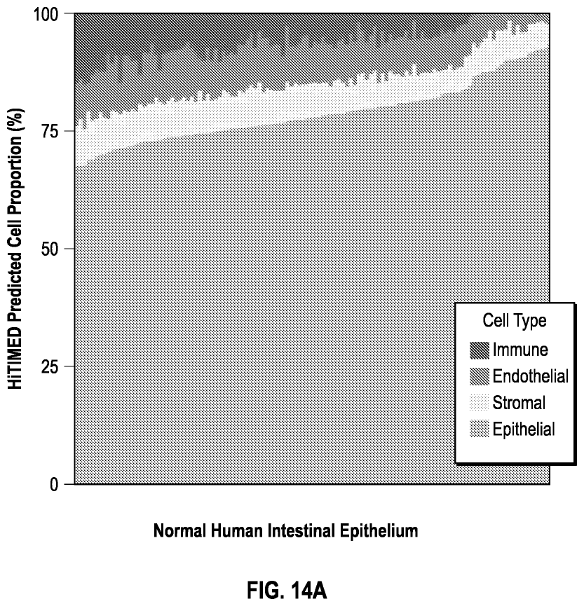

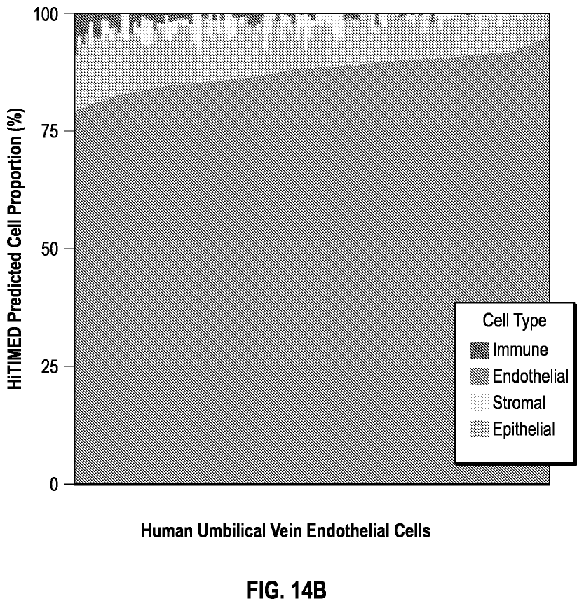

To validate HiTIMED in angiogenic microenvironment projection, publicly available purified epithelial (See Howell K J, Kraiczy J, Nayak K M, Gasparetto M, Ross A, Lee C, et al. DNA methylation and transcription patterns in intestinal epithelial cells from pediatric patients with inflammatory bowel diseases differentiate disease subtypes and associate with the outcome. Gastroenterology. 2018; 154(3):585-98.), and endothelial cells (See Franzen J, Zirkel A, Blake J, Rath B, Benes V, Papantonis A, et al. Senescence associated DNA methylation is stochastically acquired in subpopulations of mesenchymal stem cells. Aging Cell. 2017; 16(1):183-91.) are identified for HiTIMED deconvolution. In the normal human intestinal epithelium, HiTIMED predicted on average 78.7% epithelial cells (SD=6.3, (FIG. 13)). In human vein endothelial cells, HiTIMED predicted on average 87.6% endothelial cells (SD=3.6, (FIGS. 14A and 14B)).

### B. HiTIMED Deconvolution Performance Compared to Existing Methods

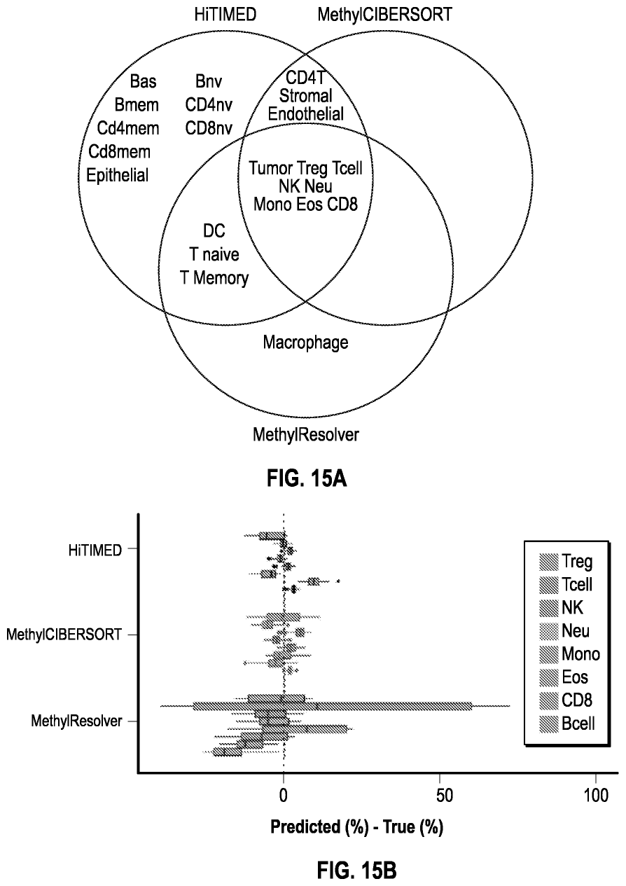

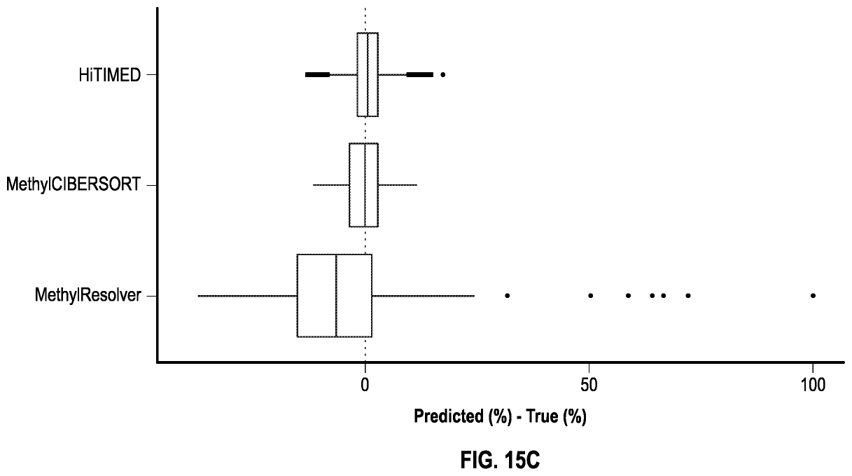

To demonstrate the advantages of using HiTIMED to deconvolve tumor microenvironment, its performance is compared with MethyICIBERSORT and Methyl-Resolver. HiTIMED encompassed all cells that can be captured by MethylCIBERSORT and MethylResolver except for macrophage and offered 8 additional unique cell types (See diagram of FIG. 15A). When comparing the performance of HiTIMED, MethyICIBERSORT, and MethylResolver on the 12 immune cell artificial mixture samples for the cell types that can be estimated by all three methods, HiTIMED show the best performance with the mean absolute error 3.54% (SD=3.3) compared to MethyICIBERSORT (Mean=3.64%, SD=2.4) and MethylResolver (Mean=15.2%, SD=16.7) (See FIGS. 15B and 15C).

### C. HiTIMED Deconvolution of Twenty Types of Carcinoma

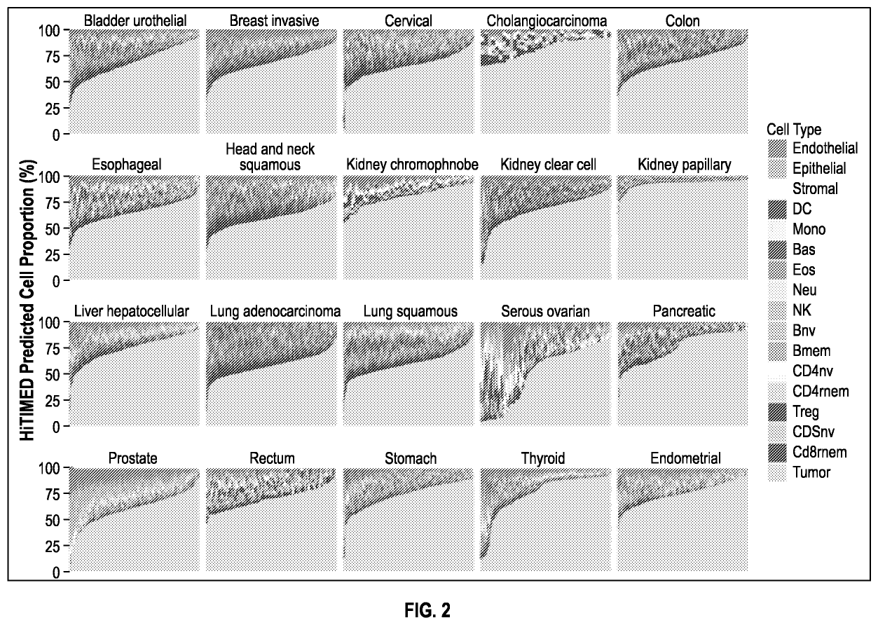

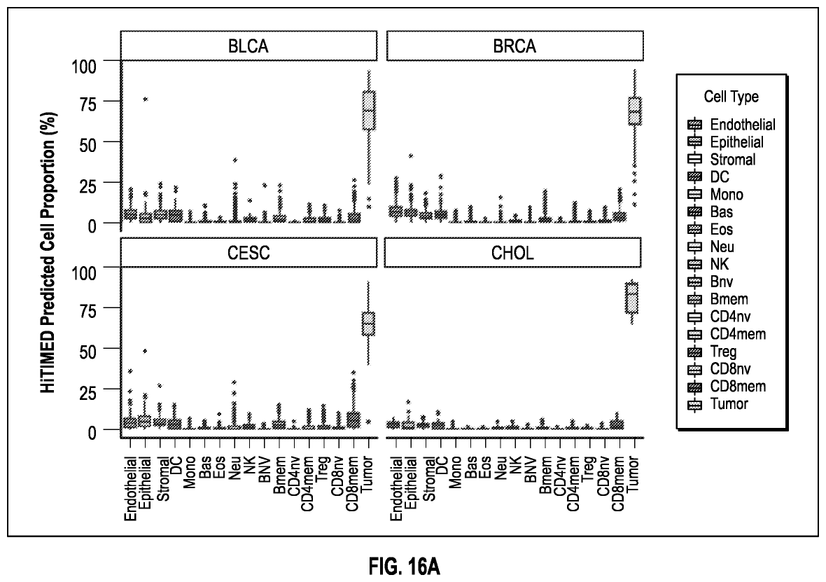

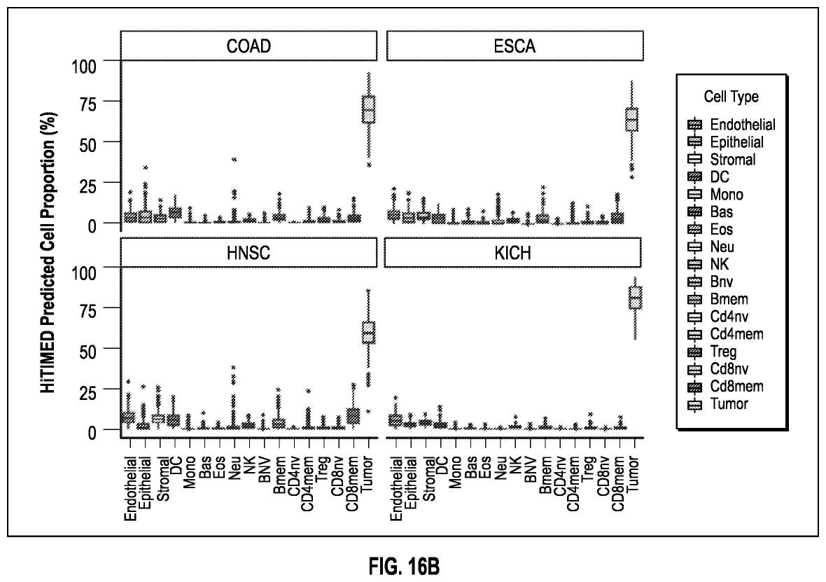

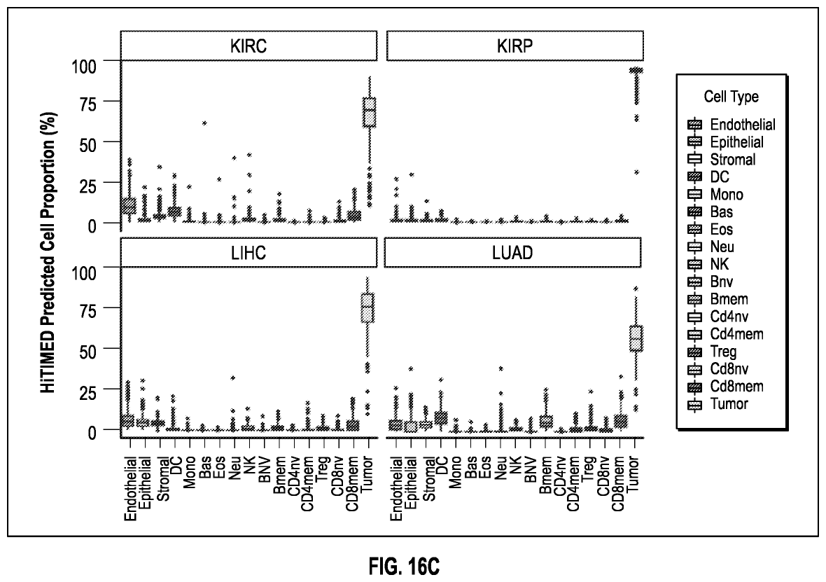

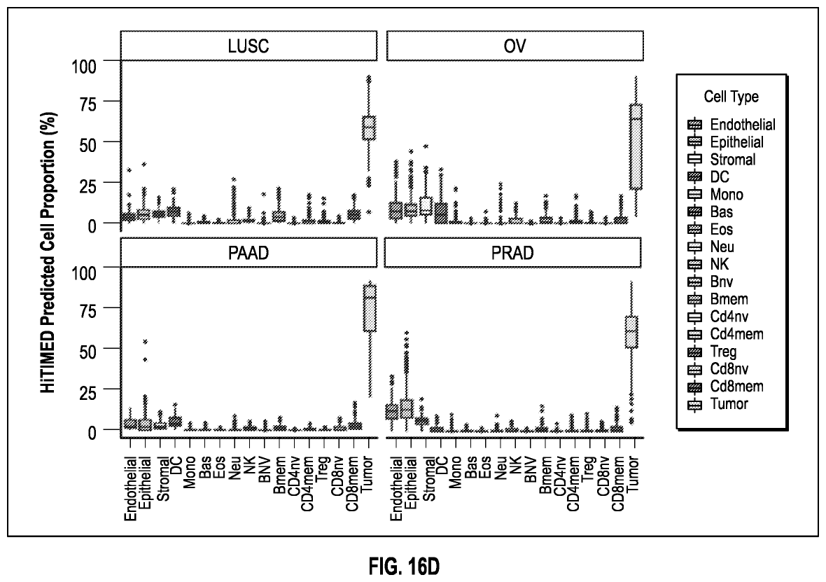

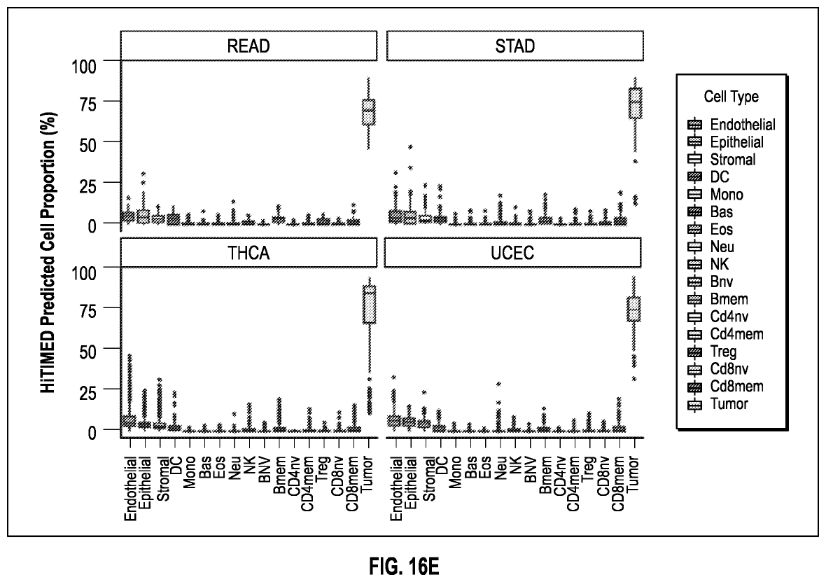

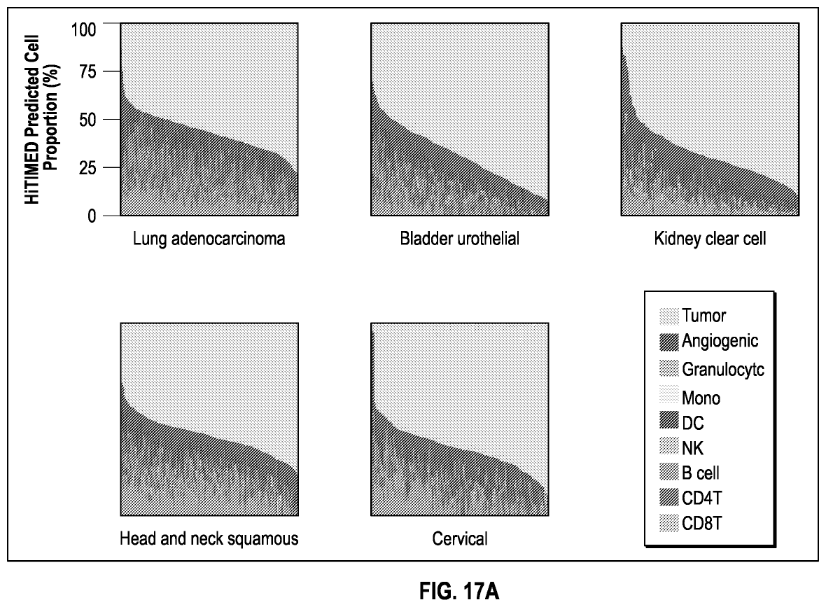

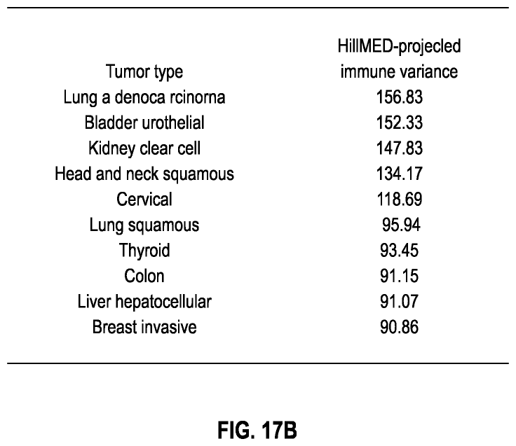

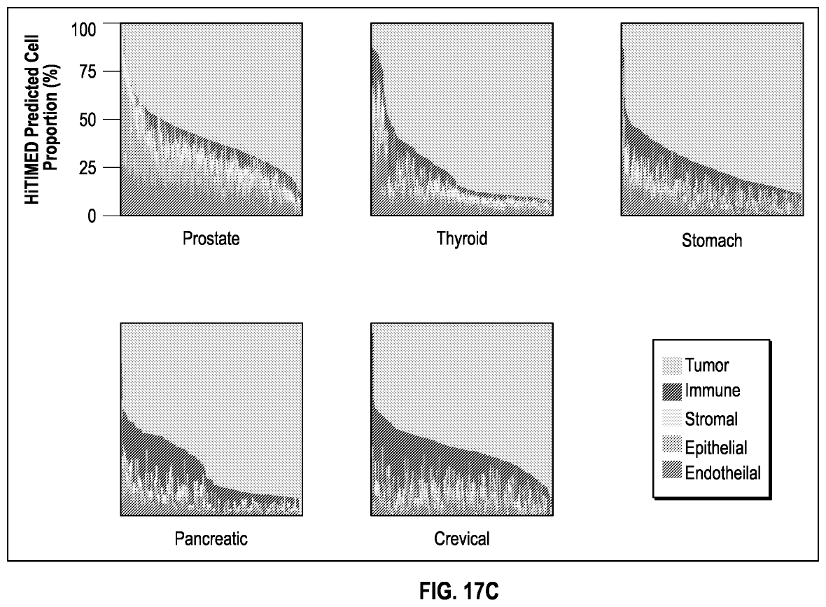

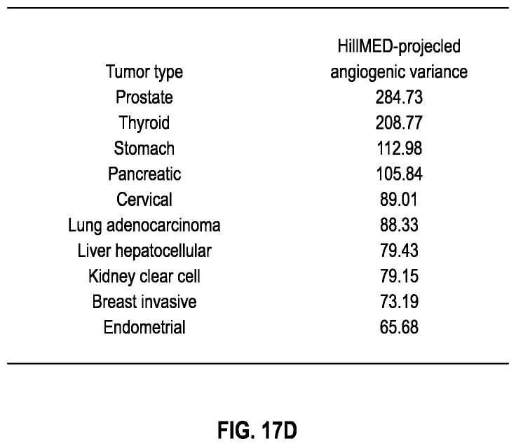

To further investigate the utility of HiTIMED, variation is identified in TME cell proportions among (e.g.) 5986 carcinoma samples from 20 tumor types using DNA methylation data from multiple sources, including TCGA and GEO. The HiTIMED projected cell proportions for each tumor are illustrated in stacked bar plots (FIG. 2) and boxplots (See FIGS. 16A-16E). Due to the limited sample size for the TCGA ovarian cancer data set, additional publicly available samples are pooled. The variation in the immune component of the TME for all tumors is assessed, and the within-tumor variation across patients in the immune component is highest in lung adenocarcinoma, muscle-invasive bladder carcinoma, kidney clear cell carcinoma, head and neck squamous cell carcinoma and cervical carcinoma (See FIGS. 17A and 17B). Assessing variation in the tumor angiogenic microenvironment uncovered the highest within-tumor variation across patients in prostate, thyroid, stomach, pancreatic, and cervical carcinomas (See FIGS. 17C and 17D). The results implied potential high variability in immune- and angiogenic-related treatment response in those tumors.

The association of specific cell type prevalence in TME with cancer patient survival is relevant to the system and method. The high resolution of HiTIMED enables us to study cell-type prevalence and survival without potential confounding by other cell types. The relationship of seven quantitatively prominent and clinically relevant immune and angiogenic cell types in TME with patients' 5-year survival is noted herein. The association of HiTIMED-projected Treg, Bmem, DC, CD8mem, epithelial, endothelial, and stromal cells, respectively, has been tested, with survival using Cox proportional hazard models adjusted for age, gender, tumor stage, HiTIMED-projected tumor proportion, and other cell-type proportions (Treg, Bmem, DC, CD8mem, epithelial, endothelial, stromal) by tumor type. Patients are stratified on the median value for each cell type. Statistically significant hazard ratios (HR) are demonstrated in the following table:

| Tumor Type | Cell Type | Hazard ratio (95% CI) |
|---|---|---|
| Head and neck squamous cell | Stromal | 1.51 (1.02, 2.22) |
| | Endothelial | 1.57 (1.07, 2.29) |
| Bladder carcinoma | Dendritic cell | 0.45 (0.28, 0.73) |
| Kidney papillary carcinoma | Endothelial | 3.48 (1.27, 9.55) |
| | Stromal | 3.44 (1.46, 8.13) |
| Lung adenocarcinoma | CD8mem | 0.50 (0.32, 0.79) |
| | Endothelial | 1.83 (1.13, 2.95) |
| | Epithelial | 0.50 (0.31, 0.80) |
| Lung squamous cell carcinoma | Epithelial | 1.80 (1.16, 2.78) |
| Thyroid carcinoma | Bmem | 7.13 (1.17, 43.53) |

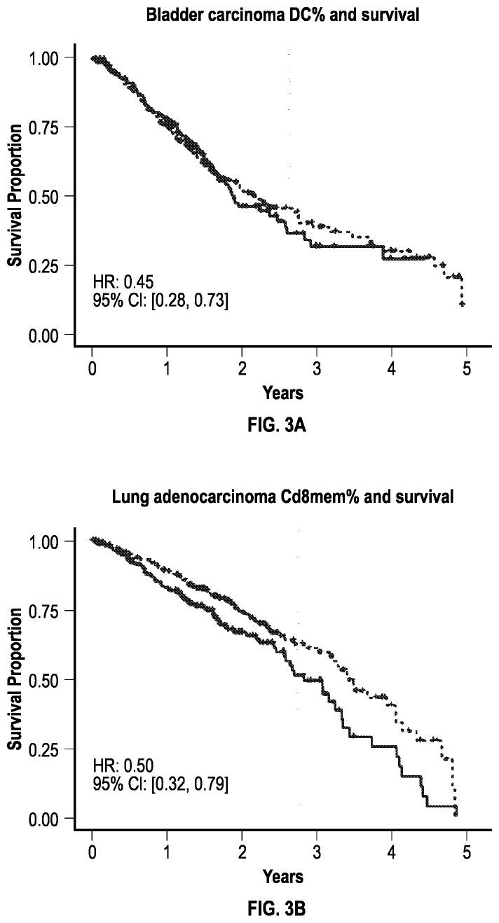

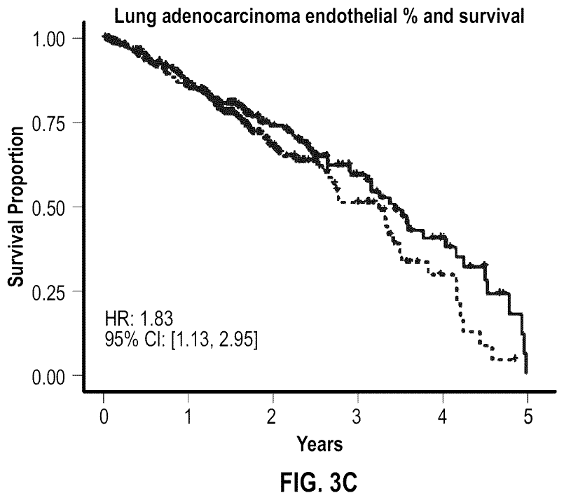

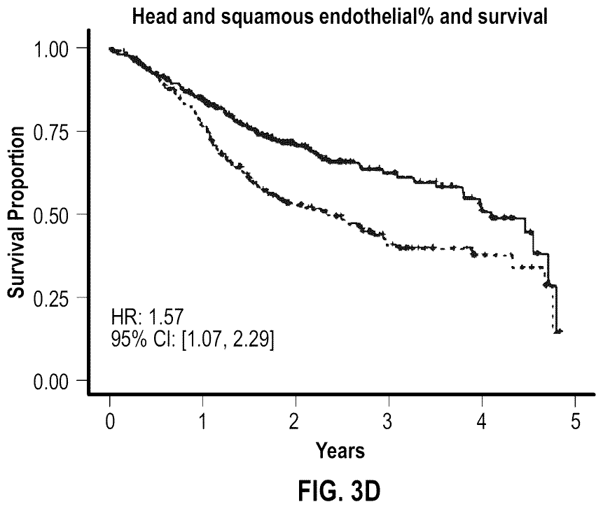

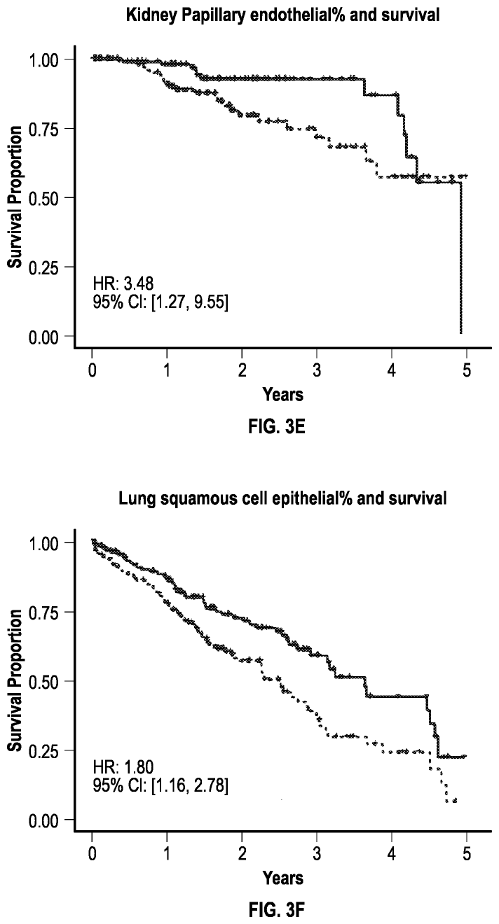

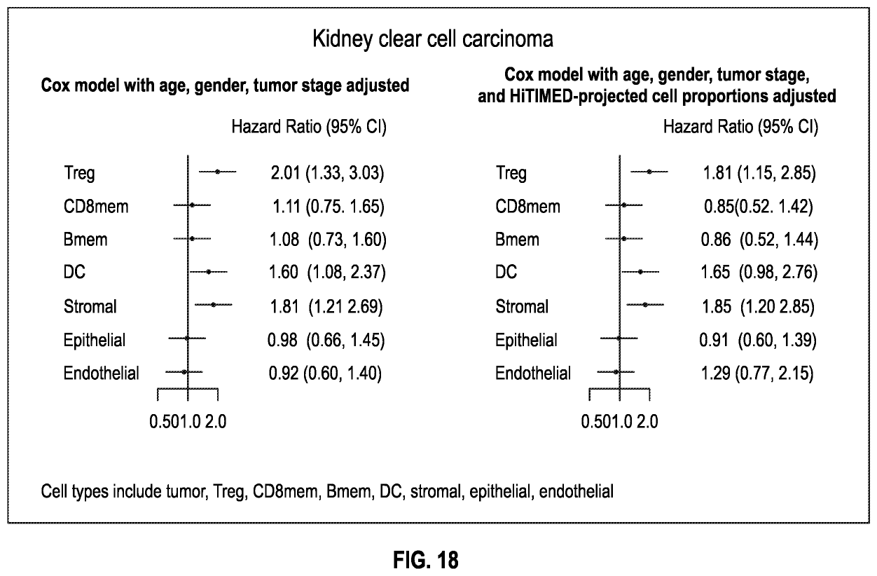

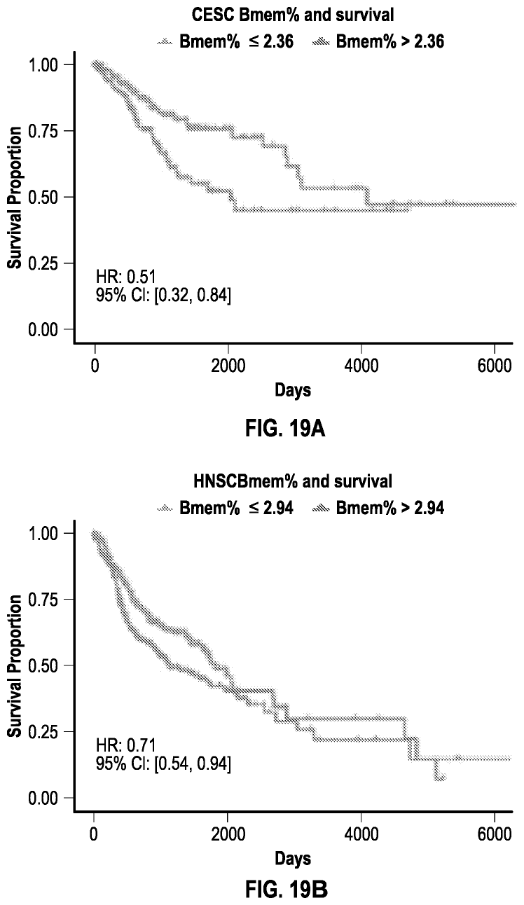

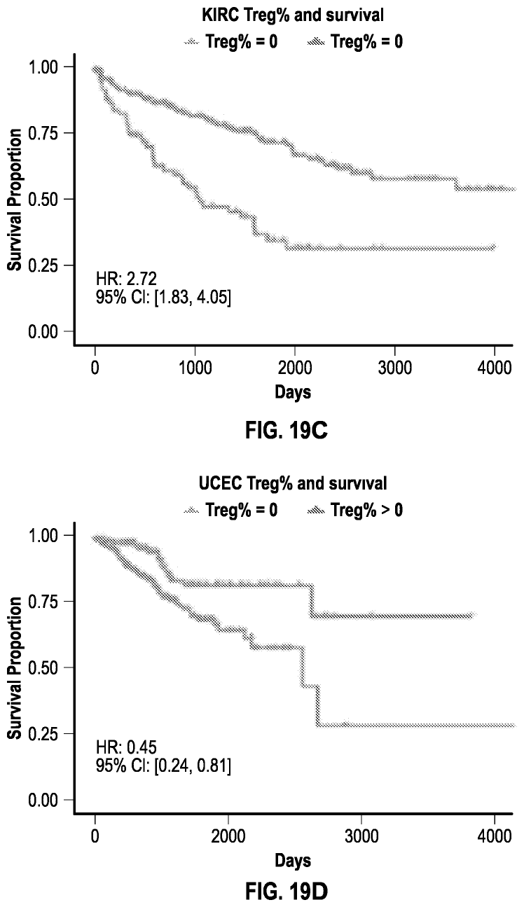

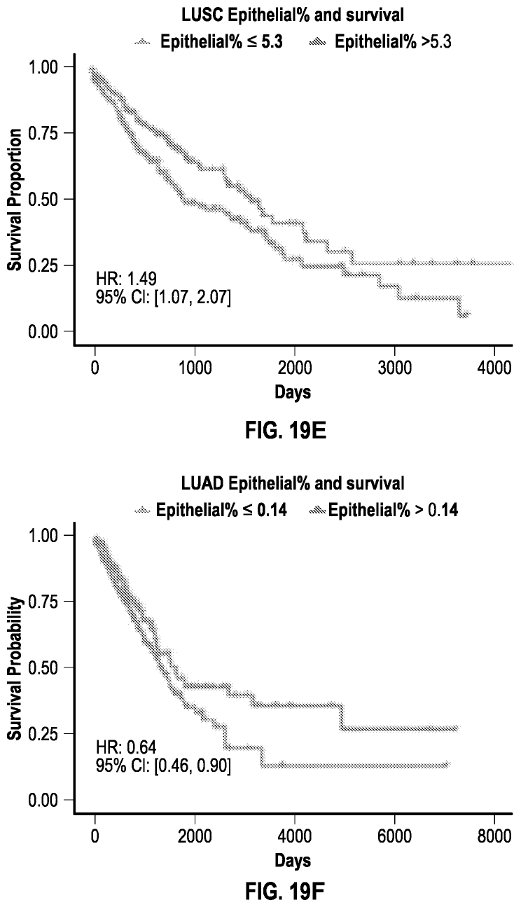

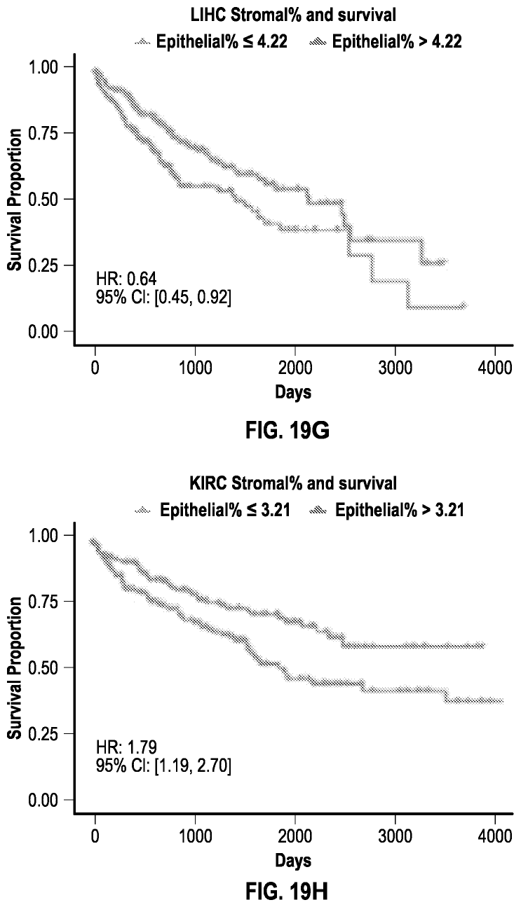

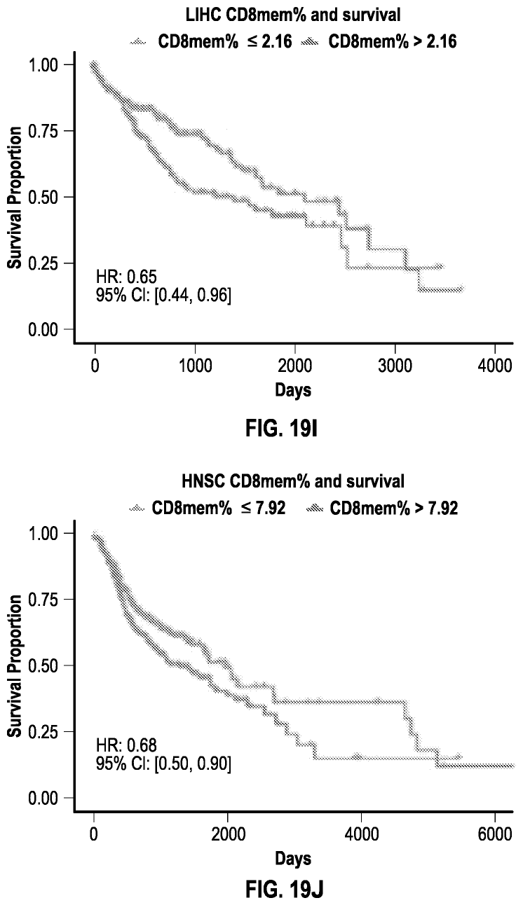

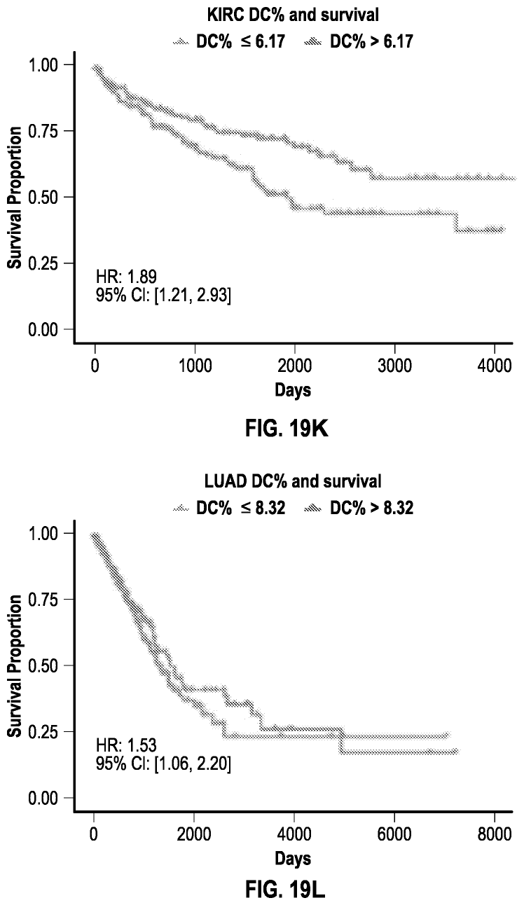

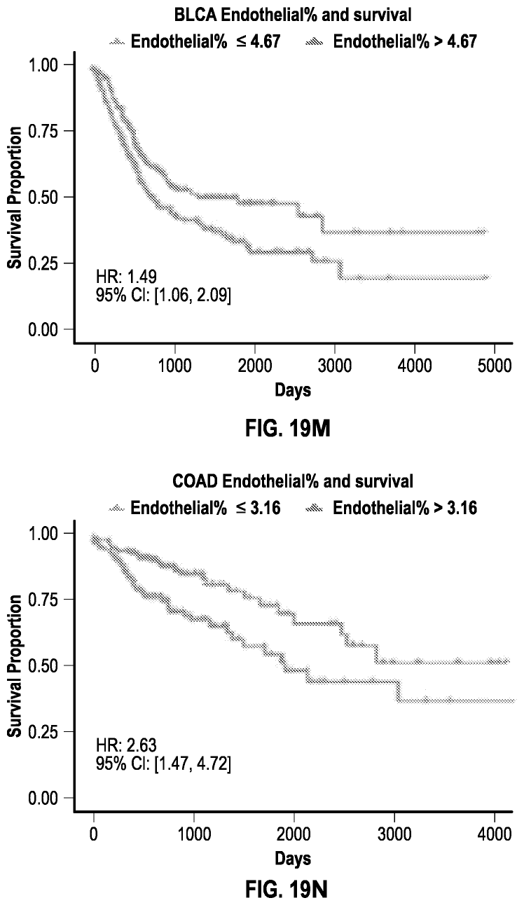

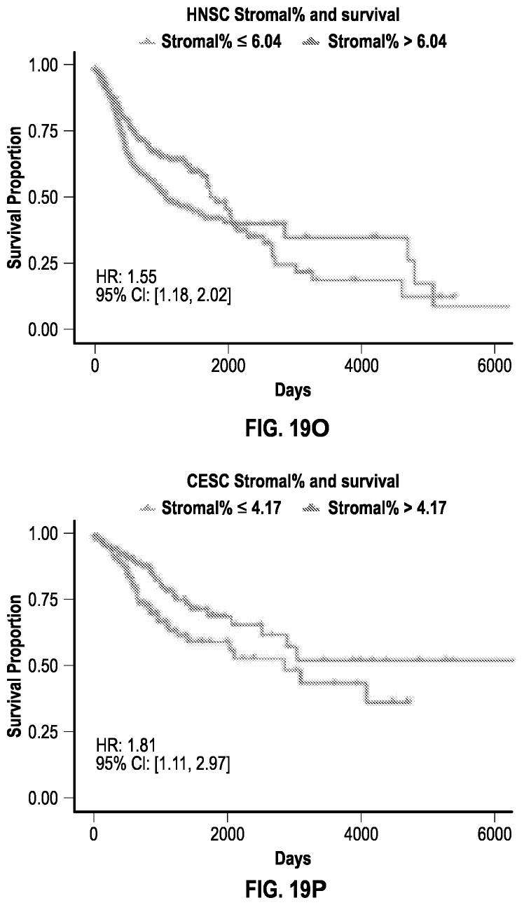

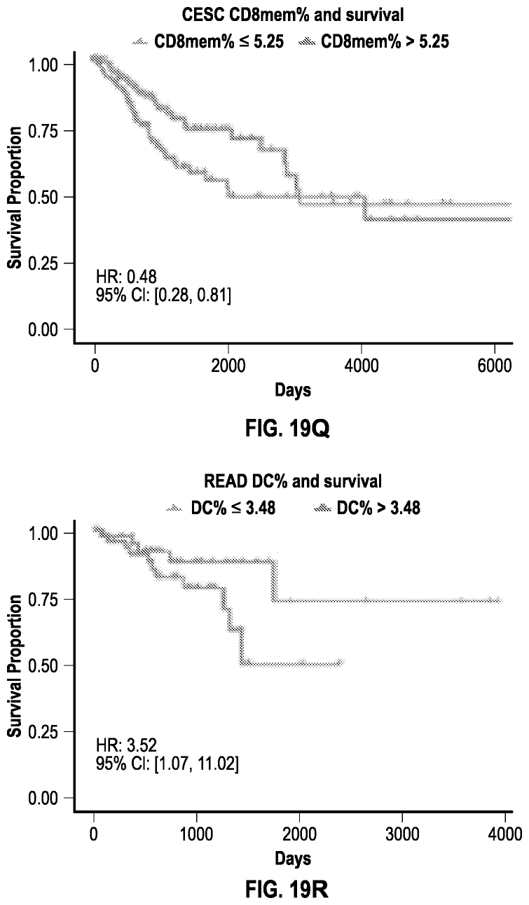

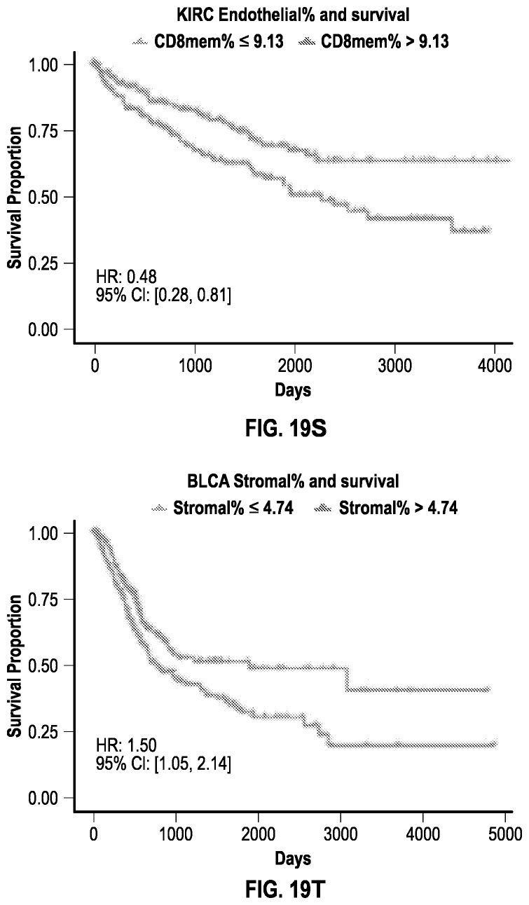

Worse 5-year survival outcomes are observed with higher than median level endothelial cell proportions in lung adenocarcinoma (HR 1.83, 95% CI [1.13, 2.95]), head and neck squamous cell carcinoma (HR 1.57, 95% CI [1.07,2.29]) (FIGS. 3A and 3D), and kidney papillary carcinoma (HR: 3.48, 95% CI [1.27, 9.55]) (FIG. 3E). In lung squamous cell carcinoma, a higher than median level epithelial cell proportion is associated with a worse 5-year survival outcome (HR 1.80, 95% CI [1.16, 2.78]) (FIG. 3F). For immune cells, better 5-year survival outcomes are observed for higher than median level DC and CD8mem proportions in bladder carcinoma (HR: 0.45, 95% CI [0.28, 0.73]) and lung adenocarcinoma (HR: 0.50, 95% CI [0.32, 0.79]) (FIGS. 3A and 3B). Note in FIGS. 3A-3F that a dashed curve represents a high value for the group, and a solid line curve represents a low value. Two Cox models in kidney clear cell renal cell carcinoma are compared with and without adjustment for cell types for a sensitivity analysis. A higher effect estimate for the association of stromal cell prevalence and survival is noted, a smaller effect estimate for the similar association of Treg prevalence and survival, and the association of the estimated DC prevalence with survival turned from significant to insignificant with survival after controlling for additional cell types (See FIG. 18). This clearly suggests that adjusting for cell types in survival analysis is crucial for both understanding the nature of these cellular interactions and interpreting their association with patient outcomes. Additional Kaplan-Meier survival curves for the significant cell proportion associations adjusting for age, gender, and tumor proportion with survival are shown in FIGS. 19A-19T.

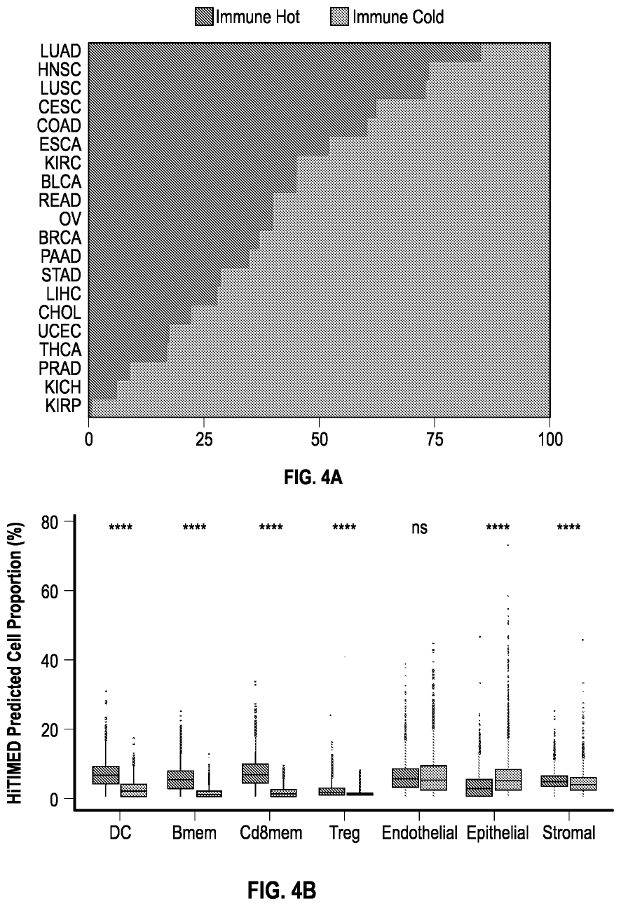

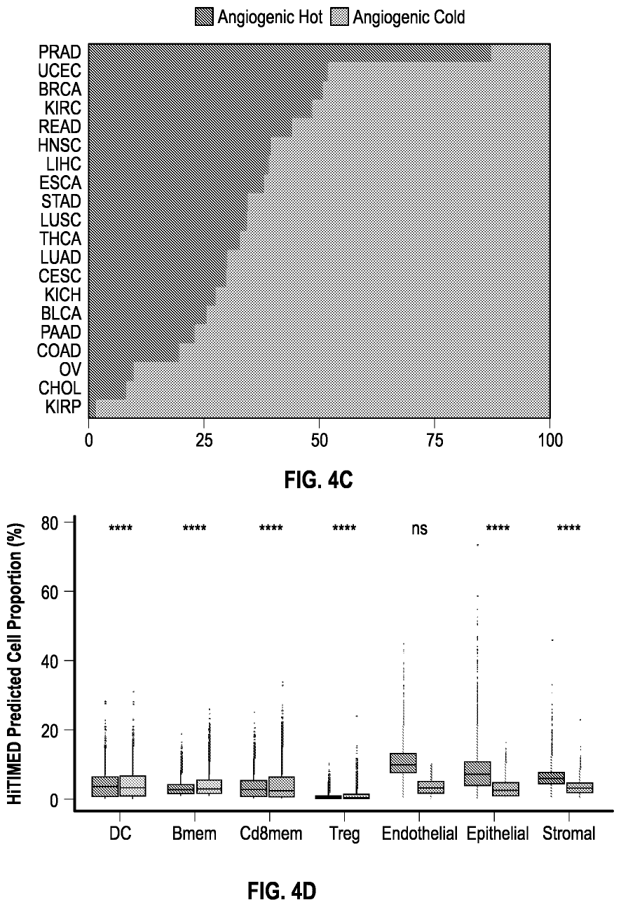

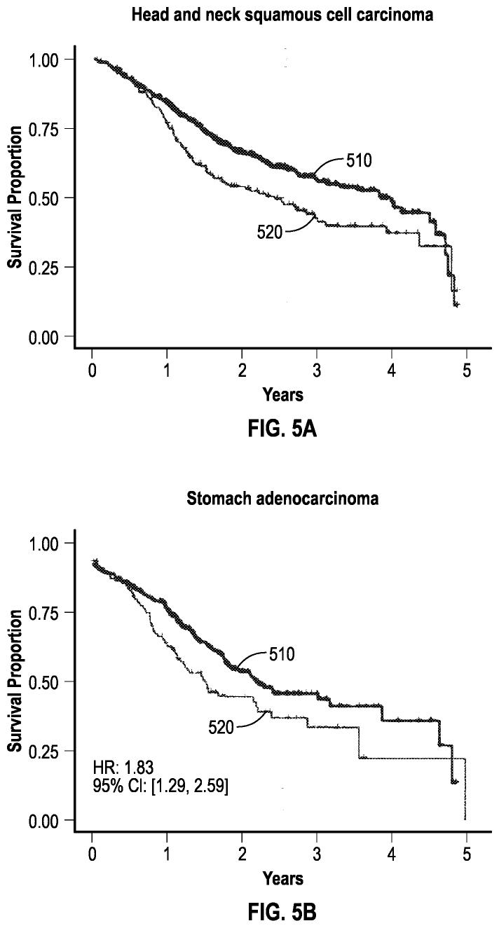

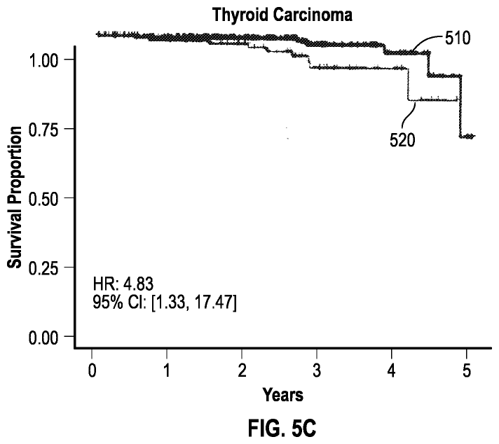

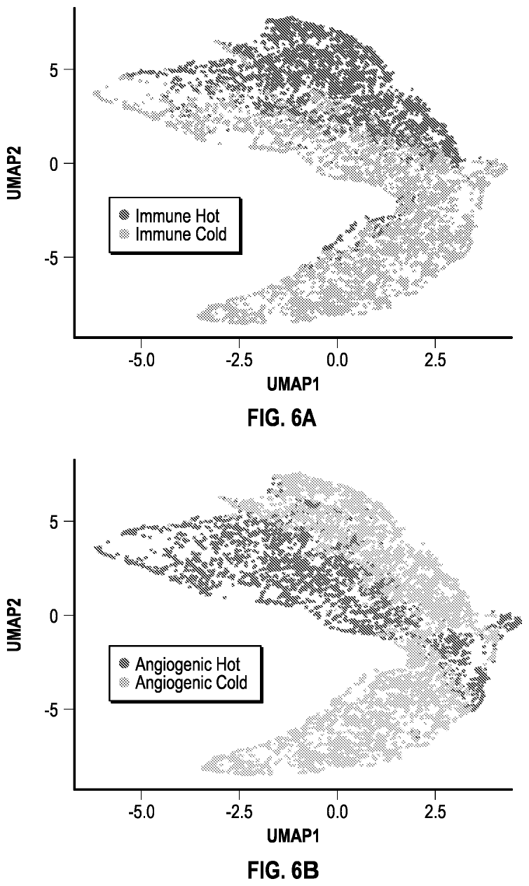

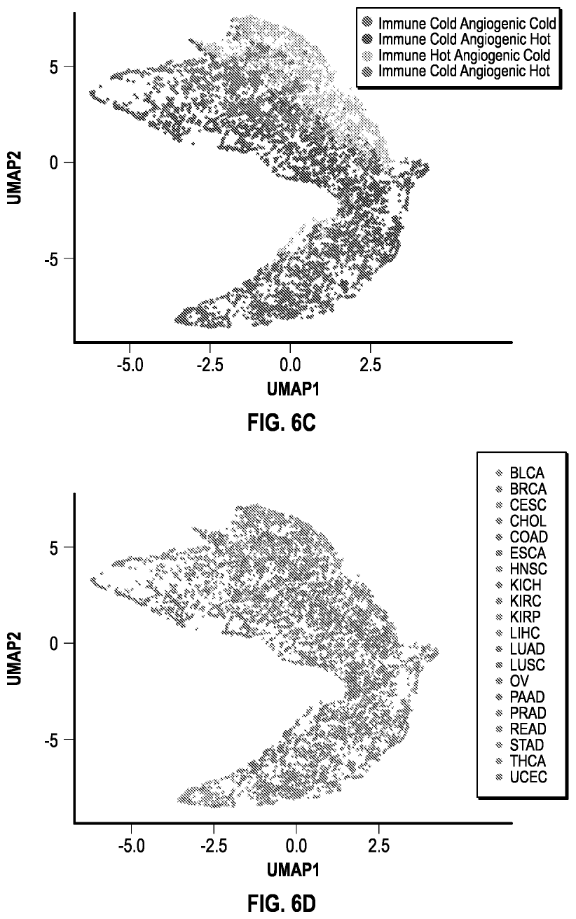

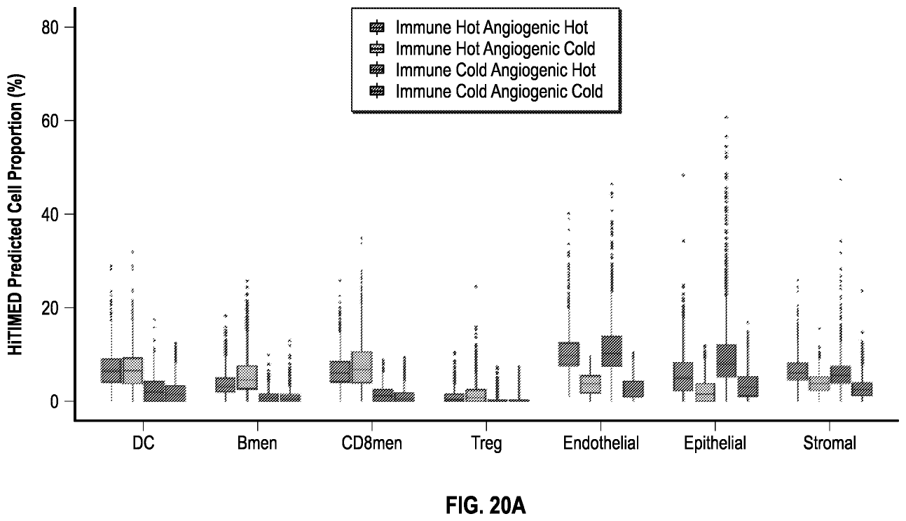

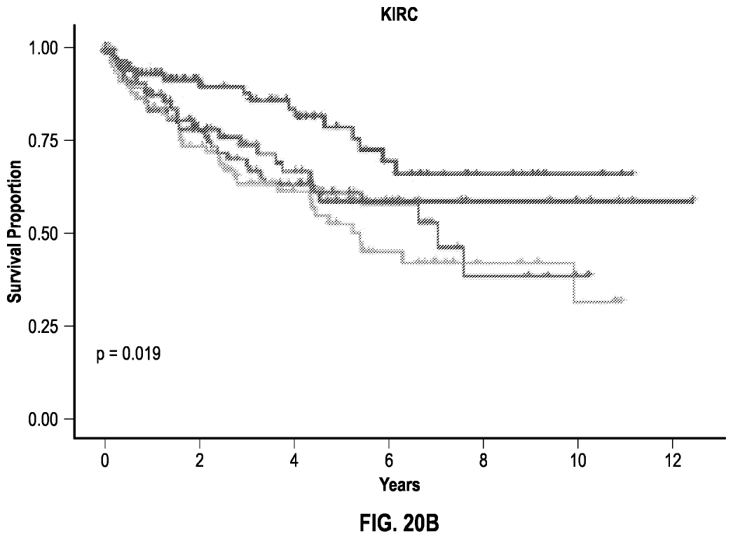

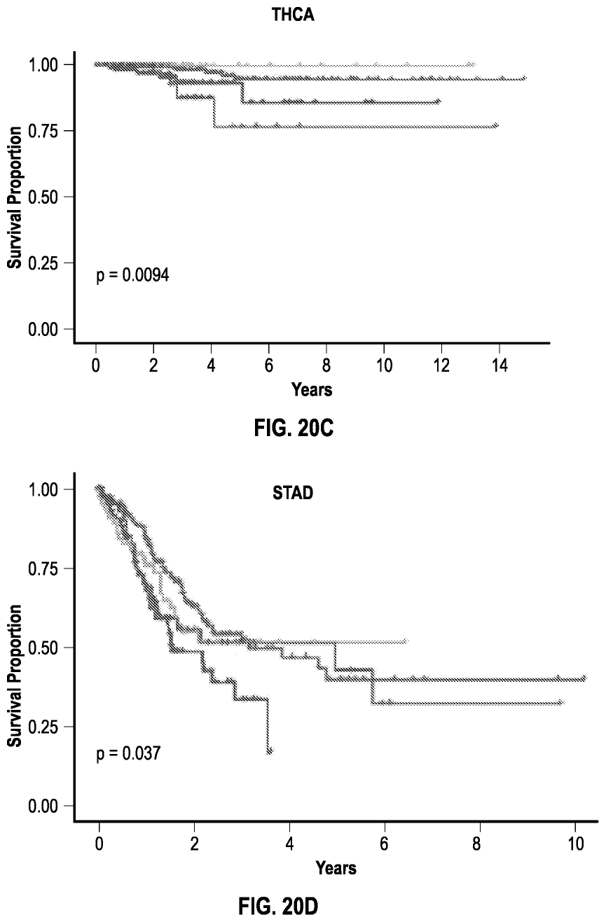

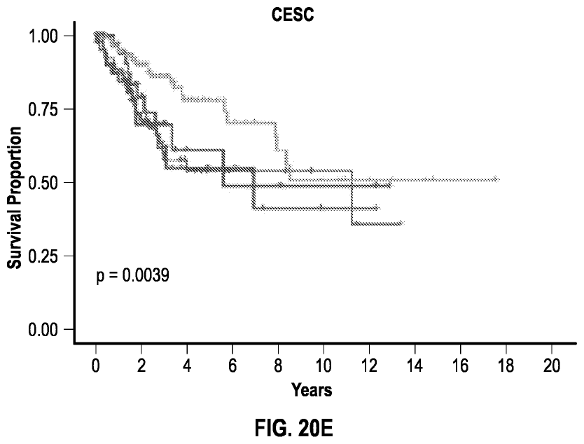

Cell profiling in TME can be used to identify tumor immune subtypes (See Thorsson V, Gibbs D L, Brown S D, Wolf D, Bortone D S, Ou Yang T H, et al. The immune landscape of cancer. Immunity. 2018; 48(4):812-30.). Previous research has used consensus partition around medoids (PAM) clustering to classify head and neck cancer immune hot and cold tumors based on predicted tumor cell fractions. Similarly, based on the HiTIMED-projected immune microenvironment compositions, the TCGA carcinomas are classified as immune hot or cold by higher or lower immune proportion in two PAM clusters (See FIGS. 4A and 4B). In the immune hot tumors, significantly higher proportions of dendritic cells (Δ=3.28%, p-value=8.5e-271), B memory cells (Δ=3.39%, p-value<2.2e-308, CD8 memory cells (Δ=5.42%, p-value<2.2e-308), and T regulatory cells (Δ=0.87%, p-value=3.4e-92) are noted, compared to immune cold tumors after adjusting for age, gender, and tumor type (FIG. 4A). The consensus PAM clustering is also employed to classify the TCGA carcinomas as angiogenic hot or cold based on the HiTIMED-projected angiogenic microenvironment compositions (See FIGS. 4c and 4D). In the angiogenic hot tumors, significantly higher proportions of endothelial cells (Δ=7.29%, p-value<2.2e-308), epithelial cells (Δ=4.12%, p-value=1.3e-221), and stromal cells (4=2.97%, p-value<2.2e-308) adjusting for age, gender, and tumor type (See FIGS. 5A-5C where curve 510 refers to angiogenic cold tumors and curve 520 refers to angiogenic cold tumors) are noted. Cox proportional hazard models are applied to interrogate the 5-year survival difference between immune/angiogenic hot and cold tumors, adjusted for age, gender, and tumor stage (FIGS. 4C and 4D). Worse 5-year survival outcomes are observed for angiogenic and neck squamous cell carcinoma (HR 1.41, 95% CI [1.05, 1.90]), stomach adenocarcinoma (HR: 1.83, 95% CI [1.29, 2.59]), and thyroid carcinoma (HR 4.83, 95% CI [1.33, 17.47]) (FIGS. 5A-5C). Four groups of tumor clusters are generated by combining the immune and angiogenic hot and cold classification (See FIG. 20A). Significantly differential survival outcomes are observed in clear cell renal cell carcinoma, thyroid carcinoma, stomach carcinoma, and cervical carcinoma across four clusters (See FIGS. 20B-20E). The UMAPs demonstrated explicit tumor clustering by immune and angiogenic hot and cold sub-types (See FIGS. 6A-6D).

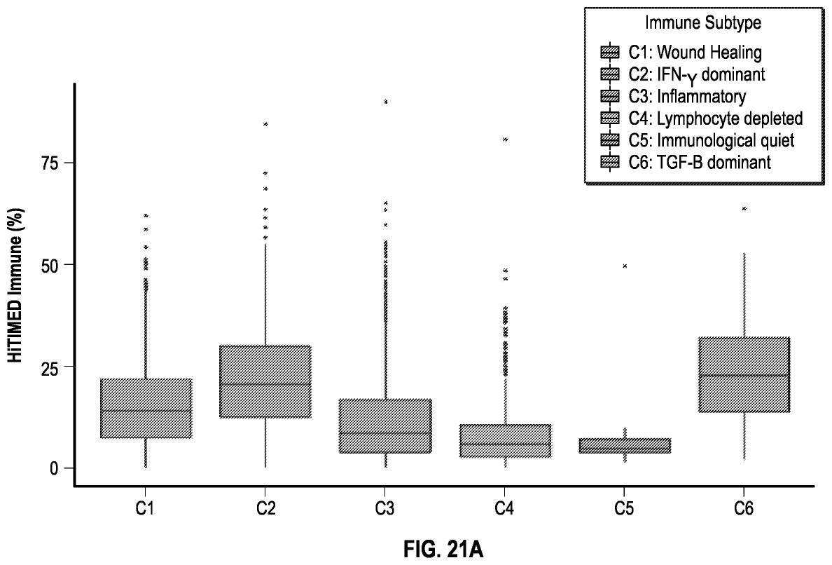

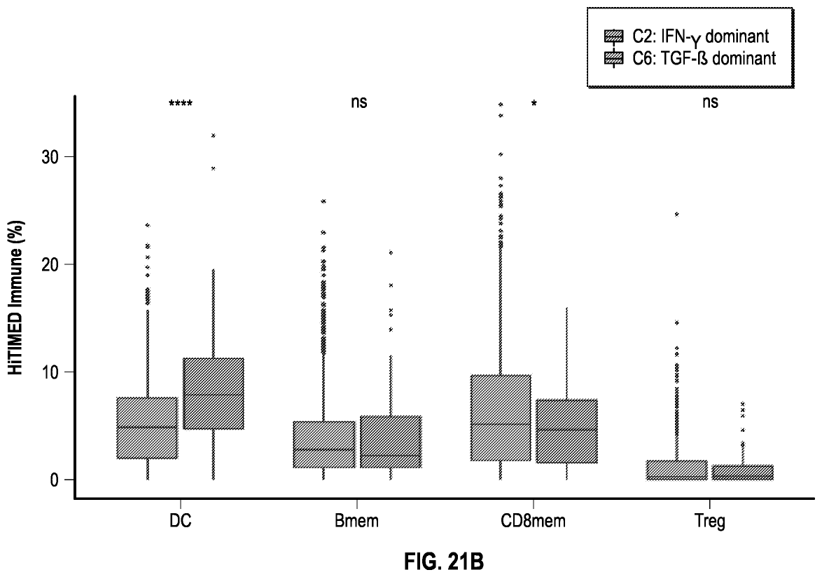

According to recent immunogenomic landscape analyses that leveraged multi-component genome-scale data sets, TCGA tumors are classified into six major immune subtypes, i.e., C1: wound healing, C2: IFN-γ dominant, C3: inflammatory, C4: lymphocyte depleted, C5: immunologically quiet, C6: TGF-β dominant. HiTIMED deconvolution shows the lowest levels of immune cells in the C4: lymphocyte depleted and C5: immunologically quiet tumors and the highest levels of immune cells in C2: IFN-γ dominant and C6: TGF-β dominant. (FIG. 21A). A Higher resolution deconvolution with HiTIMED revealed a significantly higher DC proportion (p-value=1.81e-08) and lower CD8mem proportion in C6 TGF-β dominant compared to C2 IFN-γ dominant tumors (p-value=0.016, FIG. 21B).

D. Cell-independent Tumor DNA Methylation Alterations with HiTIMED Cell Projection in Colon Cancer

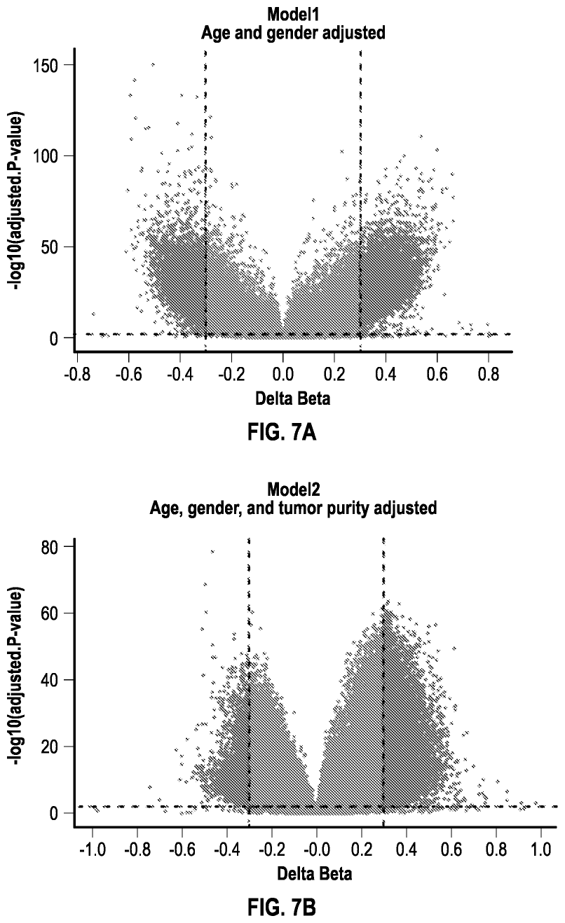

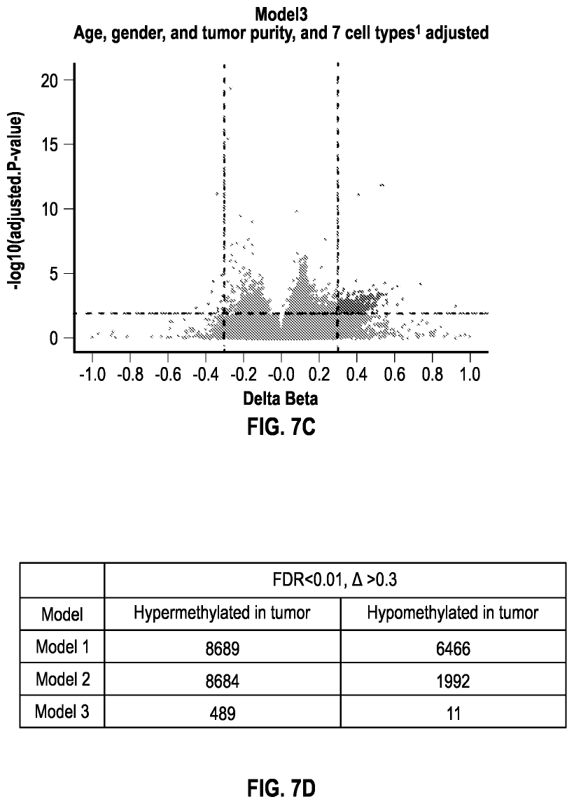

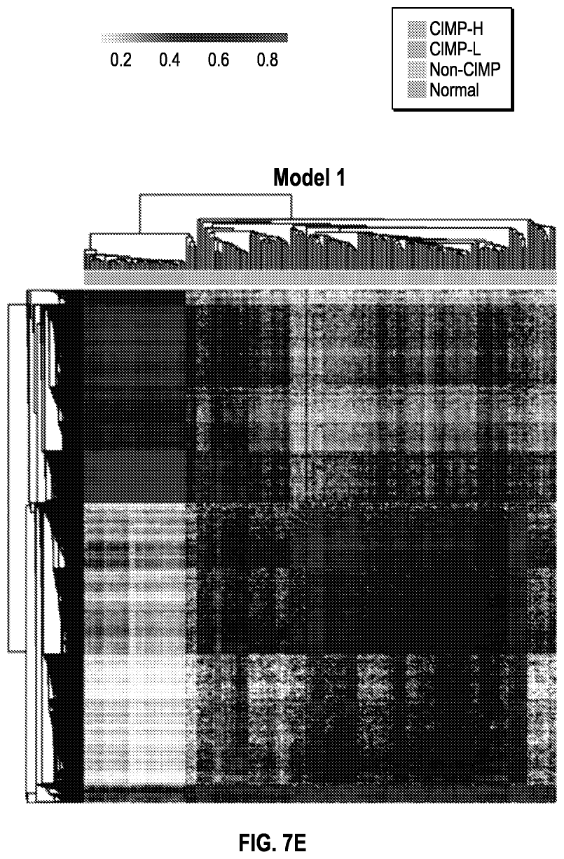

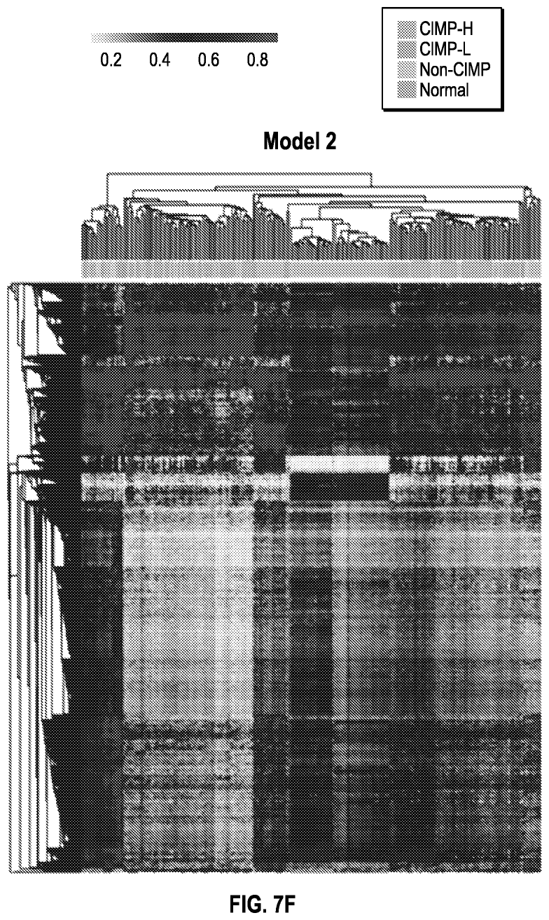

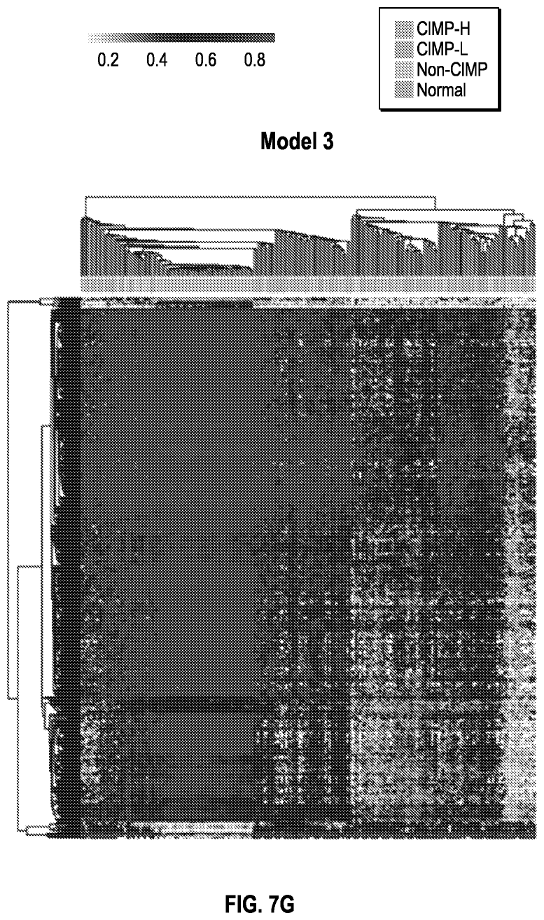

Epigenome-wide association studies (EWAS) have been widely employed on cancer to identify altered methylation patterns between cancerous and normal tissues. However, with the lack of high-resolution profiling of cell composition, current studies are typically incapable of identifying cell type-independent methylation alteration in cancer. Using HiTIMED, how a complete adjustment for TME cell composition impacts the identification of DNA methylation alterations in tumors can be established, compared with normal adjacent tissue. Models comparing methylation profiles between colon adenocarcinoma and adjacent-normal samples are tested with adjustment for age and gender and with or without adjustment for HiTIMED-projected cell proportions. Adjusting for age, gender, and eight of the most prevalent cell types resulted in a dramatic attenuation of identified CpGs with significant differential methylation in tumor versus normal tissue (Δ>0.3, FDR<0.01) (See FIGS. 7A-7D). Notably, the cell-type independent differentially methylated CpGs (DMCs) are more agnostic to the colon cancer CIMP subtypes than the DMCs identified from the unadjusted models (FIGS. 7E-7G). These results provide clear utility for isolating tumor-specific DNA methylation alterations, which has implications for basic cancer biology and developing treatment strategies.

### E. HiTIMED Deconvolution and Treatment Response

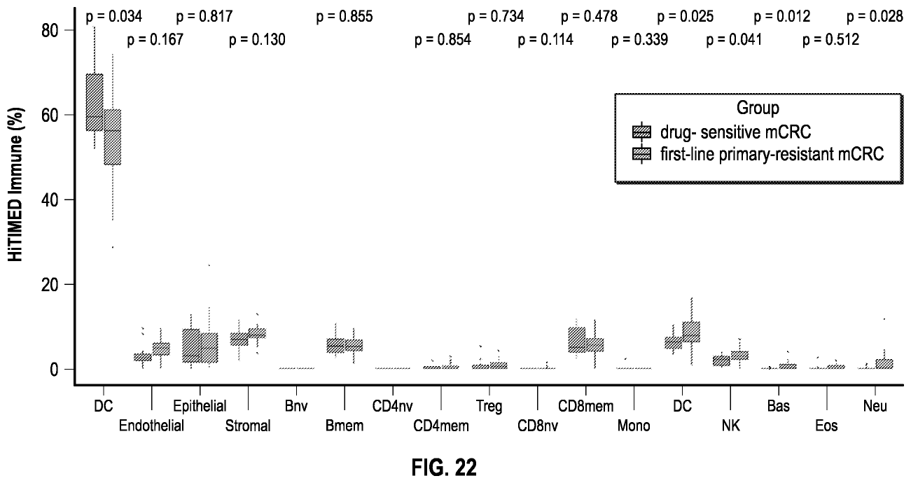

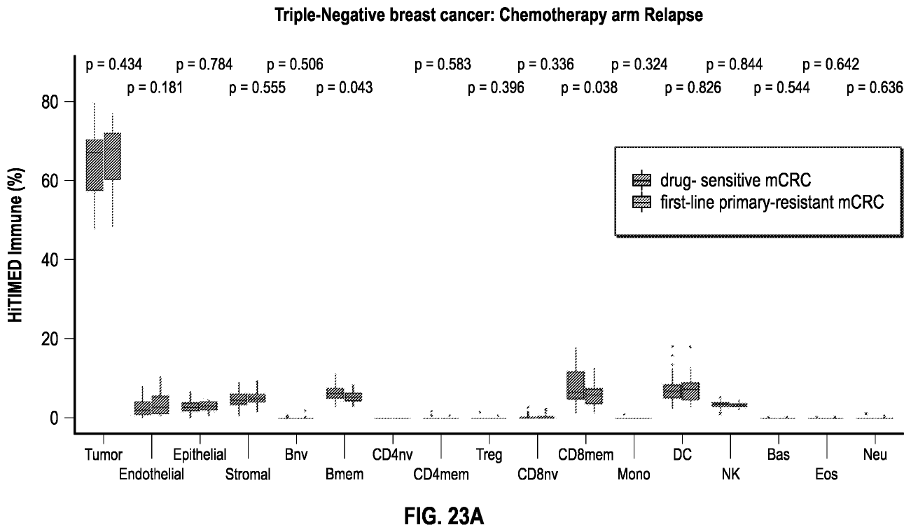

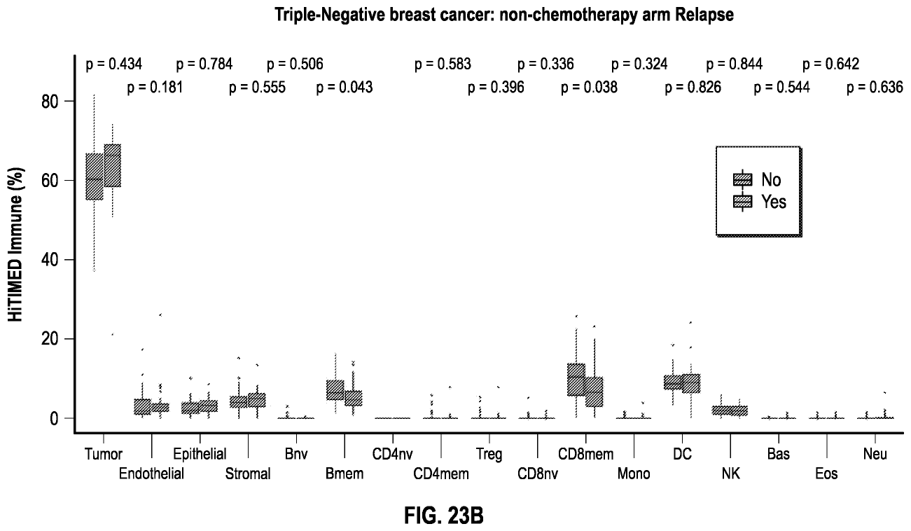

To determine how the TME is associated with treatment response, HiTIMED is applied to two publicly available data sets. One includes first-line chemotherapy drug-sensitive and -resistant metastatic colorectal cancers (mCRC). The other contains triple-negative breast cancer (TNBC) patients with and without recurrence in chemotherapy-treated and nonchemotherapy-treated arms after locoregional therapy. In mCRC, significantly lower levels of dendritic cell (Δ=2.26%, p-value=0.02), NK cell (4=1.19%, p-value=0.04), basophil (Δ=0.53%, p-value=0.01), neutrophil (Δ=1.25%, p-value=0.03) are noted, and a significantly higher tumor proportion (Δ=7.74%, p-value=0.03), in FOLFOX or FOLFIRI drug-sensitive patients compared to drug-resistant patients (See FIG. 22). In TNBC, significantly lower levels of B memory cells and CD8T memory cells are observed in relapsing tumors in both the chemotherapy treatment arm (Bmem: Δ=0.99%, p-value=0.04; CD8mem: Δ=2.18%, p-value=0.04) and the nonchemotherapy treatment arm (Bmem: Δ=1.92%, p-value=0.004; CD8mem: Δ=2.64%, p-value=0.01) (Additional file 2: Figure S16).

### F. Advantages

Previous gene expression and DNA methylation-based deconvolution approaches for TME cell composition have had some success for major cell types. However, due to the across-tumor-type diversity and within-tumor-type heterogeneity of the TME, substantial gaps still exist in tumor-type specificity, cell projection accuracy, and cell-type resolution for TME deconvolution. HiTIMED is optimized to more accurately, specifically, and exhaustively deconvolve the TME. HiTIMED has three major advantages compared to the existing algorithms: high cell-type resolution, tumor-specific libraries, and cell-projection accuracy optimization. Firstly, HiTIMED provides high-resolution profiling of the cell types in TMEs. Seventeen cell types in total among 3 TME components (tumor, immune, angiogenic) are projected by HiTIMED. In the immune microenvironment, closely related lymphocyte subtypes, including subtypes of CD4T and CD8T cells, and granulocyte sub-types are captured by HiTIMED. In the angiogenic/non-immune microenvironment, epithelial, endothelial, and stromal cells are profiled by HiTIMED separately as their roles in TME could be functionally very different. Furthermore, numerous variables from HiTIMED predicted cell types offer more opportunities to study the associations between TMEs and clinically relevant outcomes. For example, studies have demonstrated CD8mem to Treg ratio as an indicator of the immune balance between cytotoxic and regulatory immunity, corresponding to the immunotherapy response. Also, DC to NK ratio is studied in a mouse colon cancer model to enhance the antitumor effect as DC plays a crucial role in NK cell activation. The high resolution of HiTIMED projection provides novel opportunities to exploit the cellular composition of the TME to discern patient prognosis and response to therapy. Although it can be argued that single-cell RNA sequencing technologies can offer a similar resolution of cell profiling in TME, DNA methylation-based deconvolution is immensely more cost-effective, less laborious, and is amenable to archival biospecimens where cells are no longer intact. Secondly, HiTIMED uses DNA methylation signatures that are specific to tumor type. Most of the existing methods have provided a universal reference library for all types of tumors. Although, it is possible to estimate tumor purity with a signature that captures generalizable DNA methylation changes across all tumor types. The use of tumor-specific DNA methylation signatures maximizes the power of detecting most differentially methylated CpGs as tumors are genetically and epigenetically very different by tumor type. Although one algorithm has developed multiple libraries based on tumor type, cell lines are used rather than primary tumors. Studies have shown consistently differential DNA methylation profiles between cancer cell lines and primary tumor samples. Finally, HiTIMED optimizes cell projection accuracy by employing a novel hierarchical model for deconvolution. With the high resolution of cell mixture deconvolution, bias can be generated with inevitable noise for cells under similar or the same lineage. The hierarchical model enhances the projection of the primary cell types in the specific lineage niche in a stepwise manner. For example, Library L3A in HiTIMED is adapted to target angiogenic microenvironment deconvolution. As a result, the library collapses all immune cells into one group but separated epithelial, endothelial, and stromal cells for optimal discernment. Although tumor purity and major immune cells are validated for accuracy in the previously existing methods, unlike HiTIMED, extensive deconvolution of immune cell types has not been validated in other methods. Understanding the TME with a standardized and cost-effective approach enables precision medicine. Studies have demonstrated TME's association with chemotherapy and immunotherapy responses and prognosis. The balance between cytotoxic and regulatory immunity dictates tumor behavior in the immune microenvironment. When the balance favors cytotoxic immunity, tumor elimination is promoted. On the contrary, tumor escape is facilitated when the balance tips toward regulatory immunity. CD8T cells are one of the cytotoxic representatives, whereas Tregs are a proxy for regulatory immunity. Studies have shown the CD8T to Treg ratio as a significant biomarker for chemotherapy and immunotherapy responses. Analyses with HiTIMED on TCGA show better 5-year survival rates with higher CD8T memory cell levels in lung adenocarcinoma and better long-term survival in liver hepatocellular carcinoma, head and neck squamous cell carcinoma, and endocervical adenocarcinoma, which are consistent with its cytotoxic role in anti-tumoral activities. In kidney clear cell renal cell carcinoma, a higher level of Treg is associated with a worse survival outcome, indicating its role in immunosuppression. Interestingly, in endometrial carcinoma, significantly better survival with a higher level of Treg is noted. This finding is consistent with a previous report on Treg being beneficial for survival in endometrial carcinoma. The impact of Treg in cancer survival varies greatly by tumor site, suggesting differential physiological functions and roles of Tregs in different tumor types. Based on TME composition, immune hot tumors are defined as tumors with a high level of immune cell infiltration and, thus, more likely to respond to immunotherapy. The unsupervised dichotomous classification of TCGA tumors by HiTIMED immune projection demonstrates the potential identification of immune hot and cold tumors. Future supervised training on paired data on immunotherapy response with HiTIMED immune projection promises a potential on systematically rating a tumor for immunotherapy response rate.

The angiogenic microenvironment supports tumor proliferation and metastasis. The formation of new blood vessels relies heavily on endothelial and stromal cell proliferation. A higher level of endothelial and stromal cells is identified by HiTIMED is associated with worse survival rates in multiple cancers. Notably, in kidney clear cell renal cell carcinoma, a higher level of endothelial cells is beneficial for survival. This result is consistent with a single-cell analysis on kidney clear cell carcinoma, showing a better survival outcome in tumors with more endothelium. A unique role of endothelial cells in prognostication of survival and immunotherapy response in kidney clear cell renal cell carcinoma patients has been hypothesized. Worse 5-year survival outcomes are observed in multiple cancers for angiogenic hot tumors compared to angiogenic cold tumors in the analyses herein. Interestingly, immune hot and cold tumors are not significantly associated with 5-year survival after adjusting for age, gender, and tumor stage. Taken together, these data lead us to hypothesize that there is a closer relationship between the angiogenic microenvironment in TME with prognosis.

The cell type heterogeneity in TME complicates epidemiological analyses of TME and clinical outcomes. The association between cell type prevalence in TME and patient survival has previously been studied primarily by counting certain cells in TME using immunohistochemical quantification. However, the cells in TME are dynamically interactive, making such analysis susceptible to other cell type confounders. The high resolution of HiTIMED makes it possible to adjust for such cell type confounders. Further, traditional EWAS analyses are susceptible to the cell type heterogeneity confounding. For example, EWAS can identify valuable epigenetic biomarkers for early cancer detection and prognosis. However, the sensitivity and precision of identifying such biomarkers are compromised when the tissue cell heterogeneity is ignored. HiTIMED-projected cell composition in TME provides new opportunities for EWAS studies to unveil cell-type independent epigenetic biomarkers in cancer. The results herein clearly show that much of the vast DNA methylation dysregulation previously observed in tumors is attributable to cell heterogeneity. Further application of HiTIMED cell estimates to models that identify tumor-specific DNA methylation is poised to enable a clearer understanding of early DNA methylation drivers alterations in carcinogenesis and disease progression.

### G. Techniques Employed

### 1. Discovery Data Sets

For the discovery of the tumor TME deconvolution libraries, nine publicly available data sets can be used from (e.g.) TCGA, Gene Expression Omnibus (GEO), and Array Express, and two data sets from available through GEO (GSE193297, GSE167998) that contain DNA methylation microarray data on 20 types of carcinomas and their matched normal, 12 types of purified immune cell, and three types of angiogenic cell. Purified basophils, eosinophils, neutrophils, monocytes, B naïve cells, B memory cells, CD4 naïve cells, CD4 memory cells, T regulatory cells, CD8 naïve cells, CD8 memory cells are cytometric and magnetic-sorted and flow confirmed. The artificial mixtures are generated from MACS-isolated and FACS-verified cells. The cells are purchased from AllCells R: Corporation (Alameda, CA, USA), StemExpress (Folsom, CA), and STEM-CELL Technologies (Vancouver, BC, Canada). The donors include 41 males and 15 females, with a mean age of 32.2 years (sd=12.2), and multiple ethnicities, including African-Americans, East-Asian, Indo-European, and multiple/admixed. The donors are anonymous and healthy. Dendritic cells used in this study are monocyte-derived dendritic cells from healthy human blood donors. Firstly, the PBMCs are isolated from buffy coat cells by Fiscoll density gradient centrifugation. Next, the CD14 cells are purified using immunomagnetic purification. Finally, 5-day incubation with 500 U/ml human granulocyte-macrophage colony-stimulating factor (hGM-CSF) (PeproTech, Rocky Hill, NJ) and 1,000 U/ml human interleukin 4 (hIL-4) (PeproTech, Rocky Hill, NJ) completed the procedure. More details on the protocol and procedure can be found at Moss J, Magenheim J, Neiman D, Zemmour H, Loyfer N, Korach A, et al. Comprehensive human cell-type methylation atlas reveals origins of circulating cell-free DNA in health and disease. Nat Commun. 2018; 9(1):5068, and Nair S, Archer G E, Tedder T F. Isolation and generation of human dendritic cells. Curr Protoc Immunol. 2012. WorldWideWeb URL address https://doi.org/10.1002/0471142735im0732s99. Although the discovery data sets contain Illumina HumanMethylation450k or HumanMethylationEPIC array data, to ensure the applicability of the library, CpGs that are common to both platforms are retained. Furthermore, cross-reactive probes, SNP-related probes, sex chromosome probes, and non-CpG probes are masked in the analysis. 384,640 CpGs are retained after this process. The SeSAMe pipeline from Bioconductor is used to preprocess the data, including data normalization and quality control (See Hartmann B M, Thakar J, Albrecht R A, Avey S, Zaslavsky E, Marjanovic N, et al. Human dendritic cell response signatures distinguish 1918, pandemic, and seasonal HINI influenza viruses. J Virol. 2015; 89(20):10190-205.). The probes that contain over 20% of low-quality data (pOOBHA>0.05) across samples per tissue type are removed for quality control.

### 2. HiTIMED Development

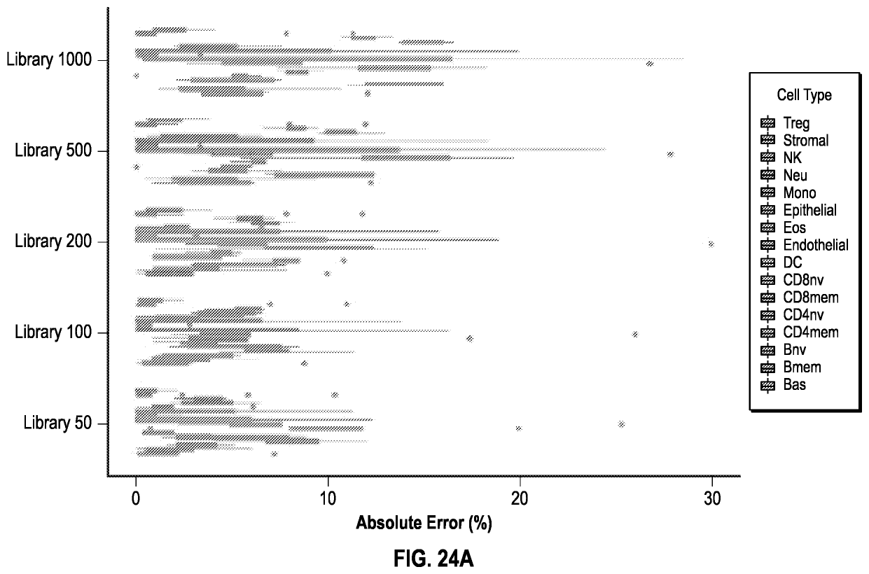

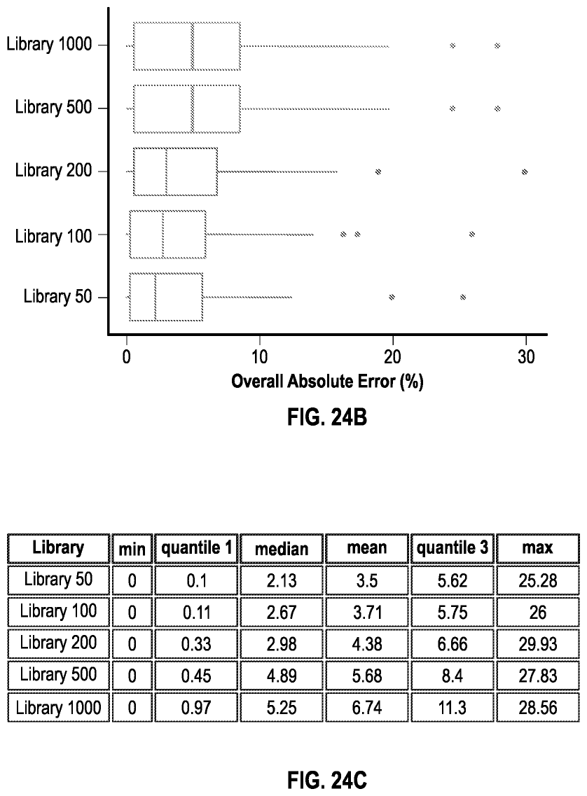

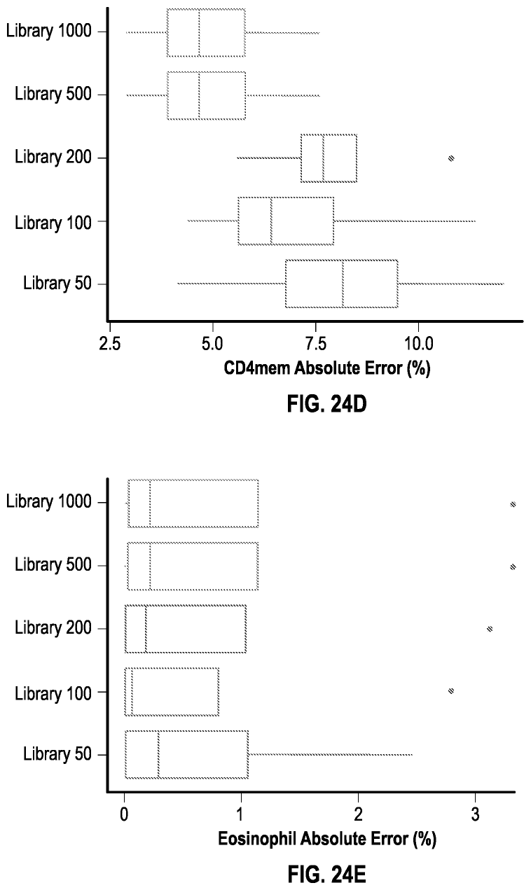

Due to the complexity and cell heterogeneity of TME, a novel, tumor-type-specific hierarchical model to develop libraries with optimized accuracy for cell projection is provided. In each tumor type, six layers of libraries are developed to hierarchically project cell proportions in first, tumor; second, angiogenic; and third, immune microenvironments (FIGS. 1A and 1B). For tumor purity estimation, the InfiniumPurify pipeline is employed to estimate the tumor purity. The method identifies the top 1000 informative differentially methylated CpG (iDMC) sites between tumor and normal samples by rank-sum test and requires that their variances of beta values are greater than 0.005 in tumor samples. The number 1000 is selected based on the performance of iterations of various numbers of iDMCs (50, 100, 200, 500, 1000, 3000, 5000, 10,000, 15,000, 20,000, 30,000, 40,000). The performance is evaluated by correlating iDMC estimated purity and ABSOLUTE purity, which is somatic copy-number-based tumor purity estimation, in lung adenocarcinoma. iDMCs are separated into hyper- and hypo-methylated groups based on their mean beta values in tumor and normal samples. The beta values for hypermethylated iDMCs remain unchanged, whereas the hypomethylated iDMC beta values are transformed to 1-beta. Density estimation with Gaussian kernel is applied to the transformed iDMC beta values. The estimated purity is the mode of the density function. More details on InfiniumPurify pipeline can be found at Zheng X, Zhang N, Wu H J, Wu H. Estimating and accounting for tumor purity in the analysis of DNA methylation data from cancer studies. Genome Biol. 2017; 18(1):17. The pipeline by identifying tumor-type-specific iDMCs is updated. Briefly, instead of using a universal set of iDMCs for estimating tumor purity for all tumor types, for each carcinoma type included in the study, iDMCs are provided specifically for that tumor type for tumor purity estimation. Epithelial, endothelial, stromal, basophil, eosinophil, neutrophil, monocyte, dendritic, B naïve, B memory, CD4 naïve, CD4 memory, T regulatory, CD8 naïve, CD8 memory cell proportions are estimated using the constrained projection/quadratic programming approach developed by Houseman et al. Houseman E A, Accomando W P, Koestler D C, Christensen B C, Marsit C J, Nelson H H, et al. DNA methylation arrays as surrogate measures of cell mixture distribution. BMC Bioinformatics. 2012; 13:86. Libraries for specific cell types have been developed using limma linear regression with empirical Bayes adjustment statistics in Meffil to reduce methylation profiles to top 100 cell-type-specific hyper- and hypo-methylated CpGs. The number 100 is selected based on the performance of iterations of various numbers of cell type-specific CpGs (50, 100, 200, 500, 1000). The performance is evaluated by calculating cell type-specific absolute error and overall absolute error in colon adenocarcinoma (See FIGS. 24A-24E). The overall absolute error is minimal when using the 50-CpG library, however, it had the worst performance in CD4 memory cell and eosinophils. To balance the performance across all cell types, the 100-CpG library is employed. More details on the hierarchical library construction are described below and in reference to FIGS. 1A and 1B.

### H. Validation of HiTIMED Projections

HiTIMED predicted tumor cell proportions have been compared to the estimated tumor purity from major existing methods, including methylation-based InfiniumPurify, MethyICIBERSORT, MethylResolver, LUMP, gene expression-based ESTIMATE, somatic copy-number-based ABSOLUTE, image stain-based IHC, and a consensus measurement of purity estimations (CPE), using TCGA tumor data. One additional data set of high-grade serous ovarian cancer is also added due to the limited ovarian cancer sample size on TCGA. Tumor type stratified comparison between HiTIMED tumor proportion and InfiniumPurify tumor purity has been conducted with Pearson's correlation coefficient, and the p-value is reported. Method paired pan-cancer tumor projection comparison is performed across HiTIMED, Methyl-CIBERSORT, MethylResolver, CPE, ESTIMATE, LUMP, IHC, and ABSOLUTE, with r and p-value reported. HiTIMED has been applied to 12 artificial mixture samples with 12 predefined immune cell proportions. RMSE, R, and p-value are calculated for each of the 12 immune cell types by contrasting the HiTIMED cell estimates versus each sample's known ground truth proportion. To validate the angiogenic/non-immune microenvironment projection, HiTIMED is applied to publicly available normal human intestinal epithelium and human umbilical vein endothelial cells. Mean and standard deviation of HiTIMED predicted endothelial proportion and epithelial proportion are reported for normal human intestinal epithelium and human umbilical vein endothelial cells respectively.

### 1. HiTIMED Deconvolution Compared to MethyICIBERSORT and MethylResolver

A Venn diagram (FIG. 15A) is shown to compare the cell types in the tumor microenvironment that can be captured by HiTIMED, MethylCIBERSORT and MethylResolver. All three methods are employed on the 12 immune cell artificial mixture samples for performance comparison. For cell types that can be estimated by all three methods, a performance comparison with operated by cell type and with all cells pooled. The error rate is calculated as PredictedProportion (%)−TrueProportion (%). The absolute error rate is calculated as PredictedProportion (%)−TrueProportion (%)|.

### 2. Statistical Analysis of the Variation of TMEs and Survival in TCGA Samples

In TCGA samples, variances of immune and angiogenic microenvironments are calculated per tumor type. Tumor types are ranked by the variance of the immune microenvironment and angiogenic microenvironment, respectively, to demonstrate the across-tumor-type variation of TMEs. Ovarian cancer is removed from this analysis due to the limited sample size with survival information. Major immune cells (Bmem, CD8mem, DC, Tregs) and angiogenic cells (epithelial, endothelial, stromal) are investigated for 5-year survival outcomes in higher than median value group compared to lower than or equal to median value group across tumors using Cox proportional hazard models with age, gender, tumor proportion, tumor stage, and other cell-type proportions (Treg, Bmem, DC, CD8mem, epithelial, endothelial, stromal) adjusted. Two Cox models, with and without cell-type adjustment, are compared in clear cell renal cell carcinoma as sensitivity analyses. Gender-specific and tumor stage information unavailable cancer types are excluded from the survival analysis. The Schoenfeld residuals are used to test the proportional hazard assumption for Cox models. To ensure that the proportional hazard assumption is not violated in the Cox models, tumor stage is stratified into high stage and low stage in lung adenocarcinoma. Age is stratified into ten groups in the bladder carcinoma data set.

### 3. Classification of Immune and Angiogenic Hot/Cold Tumors and Survival in TCGA Samples

With the high resolution of HiTIMED predicted cell types, immune and hot tumors are classified using the consensus PAM clustering method based on HiTIMED projected granulocyte, mononuclear, T cell, B cell, and NK cell proportions in TCGA samples. Similarly, consensus PAM clustering is used to classify angiogenic hot and cold tumors based on HiTIMED projected epithelial, endothelial, and stromal cell proportions. Multivariable linear regression adjusting for age, gender, and tumor type, is used to compare HiTIMED projected cell proportions between immune/angiogenic hot and cold tumors. Cox proportional hazard models with age, gender, and tumor stage-adjusted are applied to investigate the survival outcomes in immune hot vs. cold tumors and angiogenic hot vs. cold tumors. Cancer types gender-specific and with tumor stage information unavailable have been excluded from this analysis. The proportional hazards assumption of all models is checked using the Schoenfeld residuals test. Log-rank tests are used to test survival differences in four groups of tumor clusters that are generated by combining the immune and angiogenic hot and cold classification. The Student's t-test is used to compare HiTIMED immune cells between immune subtyped C2 and C6 tumors.

### 4. Models Comparing Methylation Profile Between Colon Adenocarcinoma and Adjacent Normal Samples

Three models are generated to identify DMCs between colon adenocarcinoma and normal adjacent tissues. Model 1 (FIG. 7E) adjusted for age and gender. Model 2 (FIG. 7F) adjusted for age, gender, and HiTIMED-projected tumor purity. Model 3 (FIG. 7G) adjusted for age, gender, HiTIMED-projected tumor purity, DC, CD8mem, Bmem, Treg, epithelial, endothelial, and stromal cell proportions. Delta betas larger than 0.3 and FDR smaller than 0.01 are used as the cut-off for statistically significant DMC identification. Heatmaps with Manhattan distance clustering and colon cancer CIMP subtypes colored are generated per model as depicted.

### III. Computing Environment and System and Method for Diagnosis

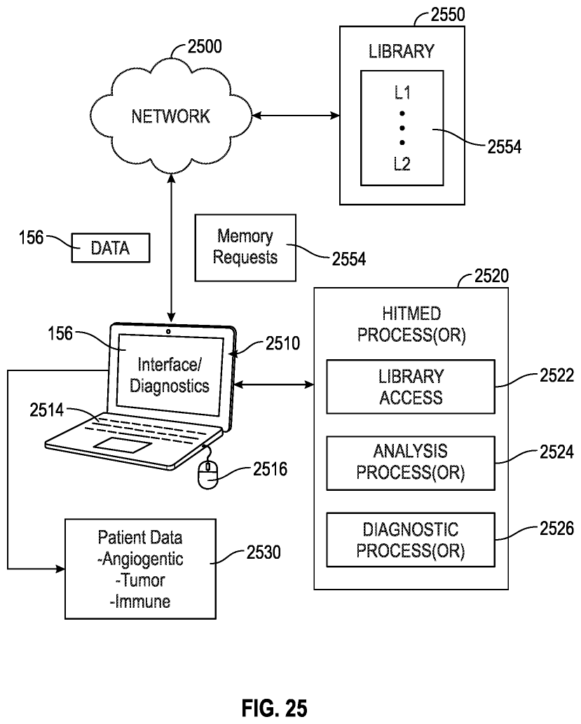

FIG. 25 shows a generalized computing environment/system 2500 for performing the tasks of the system and method herein. The system 2500 includes at least one computing device 2510 in the form of a general purpose computer (e.g., a PC, laptop, tablet, server, cloud computing arrangement, etc.) that includes an interface screen (e.g., touchscreen) 2512, and various user interface devices (e.g. keyboard 2514 and mouse 2516). The computing device instantiates a process(or) 2520 that operates the data handling and diagnostic tasks herein, as described further below. The computing device 2510 receives patient data 2530 on the cellular condition from the user via various input mechanisms—via manual input, network based-inputs from patient records and/or from appropriate medical devices. The computing device is further connected, via an appropriate wired and/or wireless link to a public and/or private data network (such as the Internet) 2540 that allows access to the layered methylation library structure 2550 described above. Access consists of requests 2554 for particular information provided in layers (L1-L6) 2552 of the library 2550, which result in the return of relevant data 2556 for use in the process(or) 2520. The library can be constructed using any appropriate data structure, including well-known database arrangements, and can be distributed among a plurality of data stores managed by one or multiple entities. Requests 2554 are directed to the appropriate store based upon a known addressing scheme.

The process(or) 2520 can be arranged in any acceptable configuration clear to those of skill, and the functional processes/ors or modules depicted are by way of non-limiting example. The process(or) 2520 includes a library access process(or) 2522 that handles patient data on conditions and user inputs to issue appropriate requests 2554 to the library 2550 and retrieve relevant data 2556. The data is used by the analysis process(or) 2524 to perform a relevant DNA methylation deconvolution on presented data. This can be facilitated by appropriate comparison routines, including those supported by commercially available (or custom) Artificial Intelligence (AI) based systems, including, but not limited to Neural Networks, Convolutional Neural Networks (CNNs), and similarly functioning systems. Such can be trained to recognize particular deconvolution patterns in the library from presented DNA samples of the patient, along with user inputs as to what type of tissue was the source of the sample. The results of the deconvolution can be presented as a diagnosis with associated data on the condition by a diagnostic process(or) 2526 using various stored and/or derived (via programmed algorithms/processes) that interoperate with results from the analysis process(or) 2524.

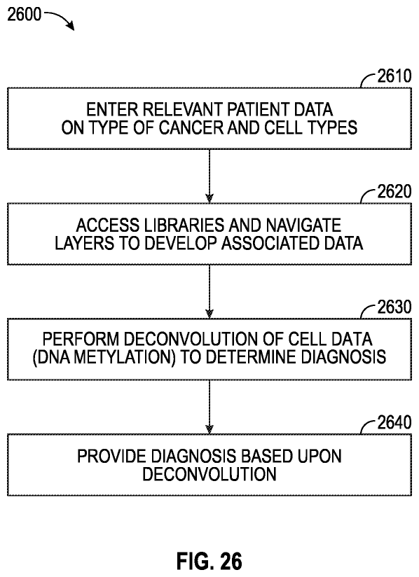

A generalized process 2600 performed by the system arrangement 2500 is shown in FIG. 26. The steps herein are shown in the overview and can more particularly draw upon the detailed library and techniques described above. In operation, relevant data is entered into the computing interface (2510) on the patient condition, including type of cancer and/or affected cells for which methylated DNA sample(s) is/are provided (step 2610). The computing system then accesses the libraries (2550) and navigates the various layers (2552) to develop associated methylation data on the input patient data (step 2620). The process 2600 then performs a DNA deconvolution of the DNA samples presented to determine relevant information, including a possible diagnosis of the condition (step 2630). Based upon the deconvolution results, diagnostic data and related information can be presented to the user in step 2640.

Notably, while the Library 2550 is established with existing data from public and proprietary sources, it is expressly contemplated that information on articular patient conditions, provided by users via the interface, can be used to establish additional data sets to one or more layers 2552 of the library. Appropriate techniques that are clear to those of skill can be employed to build the database. Likewise, the data provided can be used to further train and refine the AI based processes/ors herein to assist in identifying specific conditions via DNA methylation deconvolution.

The diagnostic and data handling services provided by the process(or) 2520 can be made available to users via a variety of techniques. For example, a secure connection, with appropriate encryption, SSL arrangements, etc. can be employed to maintain confidentiality of patient information. The service can be open source for validated users, and/or based upon a per-use charge, or subscription model.

### IV. Conclusion

It should be clear that the above-described HiTIMED, DNA-methylation-based system and method to deconvolve the TME, provides an predictable, accurate and effective technique for diagnosing and informing upon a wide range of cancerous conditions. This approach employs a novel tumor-type-specific hierarchical model with optimized libraries for each layer of deconvolution in each tumor type. HiTIMED provides higher cell type resolution compared to other methods, providing new opportunities to study the relation of the TME with etiologic factors, disease progression, and response to therapy.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments of the apparatus and method of the present invention, what has been described herein is merely illustrative of the application of the principles of the present invention. For example, as used herein, the terms “process” and/or “processor” should be taken broadly to include a variety of electronic hardware and/or software-based functions and components (and can alternatively be termed functional “modules” or “elements”). Moreover, a depicted process or processor can be combined with other processes and/or processors or divided into various sub-processes or processors. Such sub-processes and/or sub-processors can be variously combined according to embodiments herein. Likewise, it is expressly contemplated that any function, process and/or processor herein can be implemented using electronic hardware, software consisting of a non-transitory computer-readable medium of program instructions, or a combination of hardware and software. Additionally, as used herein various directional and dispositional terms such as “vertical”, “horizontal”, “up”, “down”, “bottom”, “top”, “side”, “front”, “rear”, “left”, “right”, and the like, are used only as relative conventions and not as absolute directions/dispositions with respect to a fixed coordinate space, such as the acting direction of gravity. Additionally, where the term “substantially” or “approximately” is employed with respect to a given measurement, value, or characteristic, it refers to a quantity that is within a normal operating range to achieve desired results, but that includes some variability due to inherent inaccuracy and error within the allowed tolerances of the system (e.g., 1-5 percent). Accordingly, this description is meant to be taken only by way of example, and not to otherwise limit the scope of this invention.

What is claimed is:

1. A system for determining a cancerous condition based upon at least one DNA sample of an individual comprising:
an interface arrangement that provides data related to DNA methylation for the sample, the data including related information about the sample;
a processor, responsive to the interface, that identifies the data related to the DNA methylation of the sample and accesses a data store containing a library of DNA methylation information related to each of tumor, immune, and angiogenic microenvironment components; and
a deconvolution process relative to the DNA sample and the DNA methylation information that determines association with one or more components from the sample.

2. The system as set forth in claim 1, wherein the library defines a plurality of layers of information associated with aspects of the cancerous condition relative to microenvironment components thereof.

3. The system as set forth in claim 2 wherein the one or more components define a tumor-type-specific hierarchical model related to a plurality of immune cell types that are subject to the deconvolution process.

4. The system as set forth in claim 3, wherein the deconvolution process is arranged to resolve a plurality of cell types.

5. The system as set forth in claim 4 wherein the cell types include at least one of tumor, epithelial, endothelial, stromal, basophil, eosinophil, neutrophil, monocyte, dendritic cell (DC), B naïve (Bnv), B memory (Bmem), CD4T naïve (CD4nv), CD4T memory (CD4mem), CD8T naïve (CD8nv), CD8T memory (CD8mem), T regulatory (Treg), and natural killer (NK) cells.

6. The system as set forth in claim 5 wherein the library is provided in a data store accessed over a network arrangement by the processor.

7. The system as set forth in claim 6 wherein the deconvolution process is performed by a trained artificial intelligence (AI) process.

8. A method for diagnosing and guiding the treatment of cancerous medical conditions employing results generated by the system of claim 7.

9. The method as set forth in claim 8, further comprising, treating the medical cancerous conditions based on clinical judgment of a practitioner and available therapies targeting specific cell components.

10. A method for determining a cancerous condition based upon at least one DNA sample of an individual comprising the steps of:
providing data related to DNA methylation for the sample, the data including related information about the sample;
identifying, with a processor, data related to the DNA methylation of the sample and accesses a data store containing a library of DNA methylation information related to each of tumor, immune, and angiogenic microenvironment components; and
determining, with a deconvolution process relative to the DNA sample and the DNA methylation information in association with one or more components from the sample.

11. The method as set forth in claim 10, further comprising, providing a plurality of layers of information in the library, which are associated with aspects of the cancerous condition relative to microenvironment components thereof.

12. The method as set forth in claim 11, further comprising, defining, in the one or more components, a tumor-type-specific hierarchical model related to a plurality of immune cell types that are subject to the deconvolution process.

13. The method as set forth in claim 12 wherein the deconvolution process includes resolving a plurality of cell types.

14. The method as set forth in claim 13 wherein the cell types include at least one of tumor, epithelial, endothelial, stromal, basophil, eosinophil, neutrophil, monocyte, dendritic cell (DC), B naïve (Bnv), B memory (Bmem), CD4T naïve (CD4nv), CD4T memory (CD4mem), CD8T naïve (CD8nv), CD8T memory (CD8mem), T regulatory (Treg), and natural killer (NK) cells.

15. The method as set forth in claim 14, further comprising, providing the library in a data store that is accessed over a network arrangement by the processor.

16. The method as set forth in claim 15, further comprising, performing the deconvolution process with a trained artificial intelligence (AI) process.

17. The method as set forth in claim 16, further comprising, diagnosing and guiding and treating cancerous medical conditions employing results of the step of determining.

18. The method, as set forth in claim 17, further comprising, treating the medical cancerous conditions based on the clinical judgment of a practitioner and available therapies targeting specific cell components.

19. A non-transitory computer-readable medium of program instructions, operating on the processor, that perform the steps of claim 10.

20. A non-transitory computer-readable medium of program instructions, operating on the processor, that perform the steps of claim 18.
